(19) United States
(12) Patent Application Publication
(10) Pub. No.: US 2015/0094600 A1
(43) Pub. Date: Apr. 2, 2015

(54) System And Method For Imaging Myelin

(71) Applicant: YALE UNIVERSITY, New Haven, CT (US)

(72) Inventors: Jaime Grutzendler, New Haven, CT (US); Aaron Joseph Schain, Cambridge, MA (US)

(21) Appl. No.: 14/502,506

(22) Filed: Sep. 30, 2014

Related U.S. Application Data
(60) Provisional application No. 61/885,337, filed on Oct. 1, 2013.

(51) Int. Cl. A61B 5/00

(52) U.S. Cl. CPC A61B 5/4076; A61B 5/0059; A61B 5/7425; A61B 5/4064; A61B 5/407; A61B 5/4041
USPC 600/476

(57) ABSTRACT

The present invention provides a system and method for detecting myelin and myelin-related disease. For example, the invention is based upon the finding that the combined reflectance of an administered multi-wavelength laser light specifically detects myelinated fibers. It is demonstrated herein that the present system and method effectively detects myelin, myelin defects, and myelin pathology.

### CROSS-REFERENCE TO RELATED APPLICATIONS

This application claims priority to U.S. Provisional Patent Application No. 61/885,337 filed Oct. 1, 2013, the contents of which are incorporated by reference herein in their entirety.

### STATEMENT REGARDING FEDERALLY SPONSORED RESEARCH OR DEVELOPMENT

This invention was made with government support under HL106815 and AG027855 awarded by National Institute of Health (NIH). The government has certain rights in the invention.

### BACKGROUND OF THE INVENTION

Myelin is a cellular structure that plays critical roles in action potential propagation, axonal insulation and trophic support (Nave, 2010, Nature 468:244-52) and is a potential site of experience-dependent neural plasticity (Zatorre et al., 2012, Nat. Neurosci. 15:528-36; Liu et al, 2012, Nat. Neurosci. 15:1621-3). Oligodendrocytes and Schwann cells, which are the myelin producing cells, are frequently affected in a variety of pathologies involving the brain, spinal cord and peripheral nerves (Franklin et al, 2008, Nat. Rev. Neurosci. 9:839-55; Fancy et al., 2011, Annu Rev. Neurosci. 34:21-43). These include autoimmune-inflammatory conditions such as multiple sclerosis, transverse myelitis and Guillain-Barre syndrome; inherited demyelinating conditions such as leukodystrophies and Charcot-Marie Tooth disease and acquired conditions such as cerebral ischemia, vascular dementias and diabetic neuropathy.

A variety of histological imaging techniques such as transmission electron and confocal fluorescence microscopy have been invaluable in furthering the cellular understanding of myelin development, plasticity and pathology. More recently, diffusion tensor magnetic resonance imaging (DTI) has allowed longitudinal studies of cerebral white matter tracks in animal models and humans (Bartzokis, et al., 2012, Biol. Psychiatry 72:1026-34). Cellular imaging of oligodendrocytes and Schwann cells in living organisms is also possible with genetically encoded fluorescent reporters (Kirby et al., 2006, Nat. Neurosci. 9:1506-11; Kaya et al., 2012, J. Neurosci. 32:12885-95). Furthermore, methods have been developed to image myelinated fibers without the need for fluorescent labeling including coherent anti-Stokes Raman scattering (Fu et al, 2008, Opt. Express 16:19396-409; Imitola et al, 2011, J. Biomed. Opt. 16:021109), optical coherence microscopy (Arous et al., 2011, J. Biomed. Opt. 16:116012) and third harmonic generation (Witte et al., 2011, Proc. Natl. Acad. Sci. U.S.A. 108:5970-5; Farrar et al., 2011, Biophys. J. 100:1362-71). However, the current methodologies are limited as they require complicated setups or dye injections and they have not been effectively applied to image myelin in an experimental or clinical setting because the type of information that these methods yield is limited.

Thus, there is a need in the art for improved devices and methods for imaging myelin and detecting myelin-related disorders. The present invention satisfies this unmet need.

### SUMMARY OF THE INVENTION

The present invention provides a method for imaging myelin. The method comprises administering a multi-wavelength laser light to a region of interest in a subject; collecting the reflected light from the sample, wherein the collected reflected light is indicative of myelin in the region of interest; and detecting the reflected light at each wavelength of the administered multi-wavelength laser light. In one embodiment, the method comprises constructing a plurality of single-wavelength images, each single-wavelength image based on the detected reflected light at each wavelength, and combining the plurality of single-wavelength images to form a multi-color image.

In one embodiment, the method identifies Nodes of Ranvier in a myelinated axon. In one embodiment, the method identifies Schmidt-Lanterman incisures in a myelinated axon.

In one embodiment, the region of interest is in a body tissue selected from the group consisting of brain, spinal cord, and peripheral nerve. In one embodiment, the method detects myelin at a depth of about 400 μm.

In one embodiment, the intensity of the multi-wavelength laser light is about 200-400 μW. In one embodiment, the multi-wavelength laser light comprises at least three different wavelengths. In one embodiment, the multi-wavelength laser light comprises the wavelengths of 488 nm, 561 nm, and 633 nm.

In one embodiment, the method identifies the subject as having a myelin-related disorder, such as multiple sclerosis, transverse myelitis, leukodystrophies, Guillain-Barre syndrome, diabetic neuropathy, inflammatory neuropathies, inherited demyelinating conditions, Charcot-Marie Tooth, cancer related neuropathies, or toxic neuropathies. In one embodiment, the method identifies the location of myelinated axons during surgery. In one embodiment, the method identifies a tumor as being formed of or caused by myelin producing cells. In one embodiment, the method determines the aggressiveness of a tumor.

The present invention provides a system for imaging myelin. The system comprises at least one light source, wherein the at least one light source emits a plurality of beams of light, each beam having a different wavelength; thereby generating a plurality of beams of a plurality of wavelengths; a lens for guiding the plurality of beams to a sample; and a plurality of photodetectors, wherein each photodetector is configured to detect the reflected light of one of the wavelengths of the plurality of beams.

In one embodiment, the at least one light source comprises a plurality of light sources, each configured to emit a beam of light. In one embodiment, the at least one light source is at least one of an Argon laser, a Diode Pumped Solid State (DPSS) laser, a Helium/Neon laser, a supercontinuum (white-light) laser, a laser diode, and a pulsed Ti-Sapphire laser.

In one embodiment, the system comprises an Acousto-Optical Tunable Filter. In one embodiment, the system comprises a 30/70 partially reflective mirror. In one embodiment, the system comprises a scanner. In one embodiment, the plurality of photodetectors comprise a photomultiplier tube detector.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1J, depicts the results of experiments demonstrating the in vivo imaging of mouse cortex using spectral confocal reflectance microscopy (SCoRe). (FIG. 1A) Diagram depicting the imaging and optical setup of SCoRe. Three laser wavelengths are emitted simultaneously and reflect off structures in the mouse cortex. Out of focus light is rejected by the pinhole within the microscope, and the reflected light is separated by a prism into three separate PMTs. (FIG. 1B) Representative multicolor z-projection of an image stack captured through a cranial window showing reflective fibers in layers I-II of the mouse somatosensory cortex. 50 μm scale bar. (FIG. 1C-FIG. 1F) High magnification images of monochromatic reflective signal captured with the 488 nm (FIG. 1C), 561 nm (FIG. 1D), 633 nm (FIG. 1E) lasers and then merged (FIG. 1F) to show continuous fiber detection when combined. 5 μm scale bar. (FIG. 1G) Simultaneous imaging of brain vasculature (red) with intravenous injection of fluorescent dextran and combined-wavelength image of reflective fibers (cyan) in the somatosensory cortex. Z-projection over 15 μm. 100 μm scale bar. (FIG. 1H-FIG. 1J) Z-projection of SCoRe (magenta) image stack captured from a Thy1-YFP mouse demonstrating that few YFP+ axons (green) are also reflective (arrows) however the majority of YFP+ axons and all dendrites are not reflective. 50 μm scale bar.

FIG. 2, comprising FIG. 2A through FIG. 2N, depicts the results of experiments demonstrating that SCoRe signal is dependent on myelination. (FIG. 2A-FIG. 2C) In vivo staining of cortical myelin with Fluoromyelin (FM) (red) labels only the reflective fibers (cyan). 10 μm scale bar. (FIG. 2D-FIG. 2G) In vivo FM staining in the cortex of a Thy1-YFPh mouse demonstrates that YFP positive (green), FM negative, unmyelinated axons do not reflect (arrowhead) and YFP positive dendrites similarly do not reflect (arrow), however all FM (red) positive areas are reflective (cyan). 10 μm scale bar. (FIG. 2H-FIG. 2K) Example of an axonal bifurcation in a Thy1-YFP mouse imaged in vivo demonstrating that specific parts of the same axon that are FM positive are also reflective however, the unmyelinated, FM negative portions are not reflective (arrow). 3 μm scale bar. (FIG. 2L-FIG. 2N) In vivo staining and imaging of myelin in the sciatic nerve with FM (red) reveals the location of nodes of Ranvier (arrows) which lack reflection (cyan) further demonstrating the necessity of myelin for the reflection signal. 15 μm scale bar.

FIG. 3, comprising FIG. 3A through FIG. 3M, depicts the results of experiments demonstrating the transcranial time-lapse imaging of the mouse cortex with SCoRe reveals progressive age-dependent myelination. (FIG. 3A-FIG. 3F) In vivo z-projection of SCoRe image stacks taken from the somatosensory cortex of mice throughout postnatal development demonstrating the increase in the density of myelinated axons in layers I-II even into adult stages. (FIG. 3G) SCoRe images captured through a thin skull of the same cortical region over four time points showing the appearance of new reflecting fibers. 20 μm scale bars in FIG. 3A-FIG. 3G. (FIG. 3H-FIG. 3J) High magnification z-projections captured through a thin skull of specific axons that were myelinated between imaging sessions (arrows) at the ages indicated demonstrating that SCoRe reveals new myelin formation in vivo. (FIG. 3K-FIG. 3M) Example z-projections showing repeated imaging of suspected nodes of Ranvier (FIG. 3K) (arrowheads) and myelin stability (FIG. 3L-FIG. 3M) in older animals. 10 μm scale bars in FIG. 3H-FIG. 3M.

FIG. 4, comprising FIG. 4A through FIG. 4J, depicts the results of experiments demonstrating that multicolor reflection spectrum reveals distinct myelin structures in vivo in the spinal cord and sciatic nerve. (FIG. 4A-FIG. 4B) In vivo multicolor SCoRe images captured from the spinal cord (FIG. 4A) and sciatic nerve (FIG. 4B) showing that individual fibers reflect different colors but have a predominant color consistency along each axon. 25 μm scale bars. (FIG. 4C) Two differentially reflecting axons in the sciatic nerve at high-resolution. 10 μm scale bar. (FIG. 4D-FIG. 4F) In vivo SCoRe and fluorescence images captured from an mT/mG mouse expressing tdTomato in myelin sheaths (red) (FIG. 4D) showing Schmidt-Lanterman incisures (arrows) and nodes of Ranvier (arrowheads) with detection of incisures possible via alternating line interference pattern in multicolor SCoRe signal (FIG. 4E). Combined reflection image in cyan is shown in composite with tdTomato in (FIG. 4F). 20 μm scale bar in FIG. 4D-FIG. 4F. (FIG. 4G-FIG. 4H) High magnification image of two Schmidt-Lanterman incisures showing SCoRe vertical interference pattern and fluorescent tdTomato (white) overlay (FIG. 4G) and SCoRe alone (FIG. 4H). Overlay image (FIG. 4G) generated by merging single 1 μm z sections that were 3 μm apart because the best alternating interference pattern signal comes from the top of the axon while the ideal fluorescence incisure signal comes from the middle of the axon. (i-j) High magnification SCoRe images of two Schmidt-Lanterman incisures and one node of Ranvier showing all reflected lasers (FIG. 41) and combined reflection (cyan) with tdTomato (red) (FIG. 4J). 4 μm scale bar in FIG. 4G-FIG. 4J.

FIG. 5, comprising FIG. 5A through FIG. 5L, depicts the results of experiments demonstrating that acute and chronic myelin pathology can be detected with SCoRe. (FIG. 5A-FIG. 5F) Images captured through a cranial window of P35 wildtype (FIG. 5A-FIG. 5C) and congenitally hypomyelinated shiverer mouse (FIG. 5D-FIG. 5F) showing patches of fluoromyelin (FM)-labeled myelinated axon segments (FIG. 5D) which were never seen in wildtype mice (FIG. 5A) and only the FM-labeled segments were reflective (FIG. 5E-FIG. 5F, arrowheads). 25 μm scale bar in FIG. 5A and FIG. 5D, 5 μm in FIG. 5B-FIG. 5C and FIG. 5E-FIG. 5F. In vivo SCoRe combined images (cyan) captured in a Thy1YFP mouse (white) before and after a sciatic nerve crush showing that normal axons before the crush are reflective (FIG. 5G, arrowheads) and regenerating axons that are likely not myelinated are not reflective (FIG. 5H, arrowheads). 10 μm scale bars. (FIG. 5I-FIG. 5L) SCoRe images acquired in vivo from the sciatic nerve of an mT/mG mouse with membrane bound tdTomato before (FIG. 5I and FIG. 5K) and after (FIG. 5J and FIG. 5L) intraneural injection of the demyelinating agent lysophosphatidylcholine (LPC) showing an acute change in the reflected spectrum (FIG. 5J) and in the tdTomato fluorescence distribution (FIG. 5L). 30 μm scale bar.

FIG. 6, comprising FIG. 6A through FIG. 6J, depicts the results of experiments demonstrating the imaging of cortical myelinated fibers in a human brain explant through the pial surface. (FIG. 6A) Photograph showing how images were captured from the superficial cortex of a paraformaldehyde fixed explant of human brain. (FIG. 6B) 2.5 μm z-projection of monochromatic reflective image stacks captured with the 488, 561 and 633 nm lasers, and then merged. 10 μm scale bar. (FIG. 6C) Z-projection of combined reflection image obtained from the intact human cortex explant. 30 μm scale bar. (FIG. 6D-FIG. 6E) High magnification multicolor and combined (cyan) 2.5 μm z-projections of reflection showing individual reflective fibers in the human cortex. 10 μm scale bar (FIG. 6F-FIG. 6I) High magnification multicolor and combined (cyan) images of two reflective fibers that are myelinated demonstrated by fluoromyelin labeling (red). 3 μm scale bar. (FIG. 6J) Example image of a fluoromyelin positive (red) reflective (cyan) fiber. 2 μm scale bar.

FIG. 7, comprising FIG. 7A through FIG. 7H, depicts the results of experiments demonstrating the simultaneous in vivo SCoRe (cyan) and confocal fluorescence images captured through a cranial window after topical application of sulforhodamine 101 (red) to label astrocytes in a Thy1YFP (green) (FIG. 7A-FIG. 7D) or CX3CR1GFP transgenic mouse (FIG. 7E-FIG. 7H) demonstrating implementation of SCoRe with confocal fluorescence imaging in vivo and also showing that astrocytes, dendrites, and microglia are not reflective. 30 μm scale bars.

FIG. 8, comprising FIG. 8A through FIG. 8J, depicts the results of experiments demonstrating that high-zoom fast time-lapse imaging with SCoRe induces virtually no photobleaching of fluorescent structures. In mouse cortex which has been labeled with Fluoromyelin Red (FM), the reflection (FIG. 8A) and FM fluorescence (FIG. 8B) can be seen to co-label. In spot (i), high speed reflectance imaging was conducted for 5 minutes, and in spot (ii) high speed fluorescence imaging was conducted for 5 minutes. After imaging, the reflectance signal in both spots is unchanged (FIG. 8C), while the FM fluorescence signal (FIG. 8D) is heavily photobleached in spot (ii), but unchanged in spot (i). Images from the first (FIG. 8E and FIG. 8G) and last (FIG. 8F and FIG. 8H) frames of the reflectance in spot (i) (FIG. 8E-FIG. 8F), and fluorescence of spot (ii) (FIG. 8G-FIG. 8H) during high speed imaging. Photobleaching was quantified in (FIG. 8J) as a percentage of the intensity of reflectance or fluorescence after 5 minutes of imaging as compared to the intensity before imaging. Note that reflectance imaging for 5 minutes does not affect reflectance or fluorescence intensity, while fluorescence imaging for 5 minutes bleaches the FM by approximately 40% but does not affect the reflectance image (n=4, *p<0.00001 to all other groups using 1-way ANOVA).

FIG. 9, comprising FIG. 9A through FIG. 9G, depicts the results of experiments demonstrating individual cortical oligodendrocytes imaged in vivo at P21 with SCoRe. (FIG. 9A) Low magnification SCoRe image captured through a cranial window showing patches of reflective fibers with specific domains. 100 μm scale bar. (FIG. 9B-FIG. 9G) High magnification images showing individual patches of reflective fibers with morphology reminiscent of individual mature oligodendrocyte territories. 50 μm scale bars.

FIG. 10, comprising FIG. 10A through FIG. 10F, depicts the results of experiments demonstrating that axial rotation does not affect myelinated fiber reflected spectrum. A sciatic nerve was imaged with SCoRe (FIG. 10A), and neighboring axons are shown with their approximate color related to their reflected spectrum above. The nerve was then rotated axially and re-imaged (FIG. 10B). Some axons are obscured by epineurium reflection after the rotation (bright surface reflection). The same axons can be seen in orthogonal views (FIG. 10C and FIG. 10D, respectively). The axons retain their reflective spectrum even though their relative positions to other axons, the connective tissue, and the lens has changed, proving that these relative positions are not the determining factor for reflected spectrum. In (FIG. 10E-FIG. 10F), a diagrammatic representation of the change in position of the axons relative to the objective lens is shown.

FIG. 11, comprising FIG. 11A through FIG. 11C, depict the results of experiments demonstrating that mT/mG mice and fluoromyelin can be used to visualize different features of myelin structure. Sciatic nerves of mT/mG mice were injected in vivo with Fluoromyelin (FM, green), which labels compact myelin, and a single myelinated axon near a node of Ranvier (star) is shown. The tdTomato (FIG. 11A) can be seen most brightly in cytosolic compartments of the Schwann cell, namely the inner (little arrow) and outer (arrowhead) cytoplasmic tongues, along with Schmidt-Lanterman incisures (large arrows). FM (green) (FIG. 11B) binds to compact myelin and can be seen in composite (FIG. 11C) to fill the space between the inner and outer cytoplasmic tongues labeled by tdTomato (red), but does not highlight Schmidt-Lanterman incisures in contrast to mTmG mice. 10 μm scale bar.

FIG. 12, comprising FIG. 12A through FIG. 12L, depicts the results of experiments demonstrating that myelin pathology can be detected with SCoRe in the peripheral nerve and spinal cord. (FIG. 12A and FIG. 12D) Low magnification images of YFP+ axons in the sciatic nerve of a Thy1YFP mouse before (FIG. 12A) and 7 days after (FIG. 12D) nerve crush (showing low magnification images of the same nerves in FIG. 5). 100 μm scale bars. (FIG. 12B-FIG. 12C and FIG. 12E-FIG. 12F) High magnification images taken from the regions depicted in the boxes in FIG. 12A and FIG. 12D showing combined reflection before (FIG. 12B-FIG. 12C) and after (FIG. 12E-FIG. 12F) nerve crush showing that regenerating non-myelinated axons are not reflective (arrowheads in FIG. 12E-FIG. 12F) (same images shown in FIG. 5). 10 μm scale bar. (FIG. 12G-FIG. 12H) SCoRe image captured from an acute spinal cord explant before (FIG. 12G) and after (FIG. 12H) exposure to a hypotonic solution (H₂O) demonstrating a change in the reflected spectrum after 20 minutes. 70 μm scale bar. (FIG. 12I-FIG. 12J) In vivo sciatic nerve SCoRe images showing a change in the reflected spectrum 30 minutes after exposure to DMSO which changes the phospholipid composition of the membrane. 15 μm scale bar. (FIG. 12K-FIG. 12L) In vivo sciatic nerve SCoRe images from a Thy1YFP (yfp=white) showing a change in the reflected spectrum 20 minutes after exposure to a hypotonic solution. Note that the YFP axon was not substantially changed during this interval, demonstrating that SCoRe is very sensitive to early injury. 10 μm scale bar.

FIG. 13, comprising FIG. 13A through FIG. 13F, depicts a hypothetical reflection mechanism and evidence of the influence of myelin thickness on reflection spectrum. (FIG. 13A-FIG. 13D) Examples of a pulled glass electrode and its reflective spectrum. Glass cylinders were pulled using a P97 Sutter glass pipette puller so that they decrease in size over distance. Glass electrodes of thickness around 1-2 μm, similar to an axon, were imaged with SCoRe (FIG. 13A and FIG. 13C), and the relative fluorescence intensities of the blue, green, and red reflection were graphed along the length of the electrode (from top to bottom, with the electrode decreasing in size) (FIG. 13B and FIG. 13D respectively). (FIG. 13E) A diagram of the properties of the thin film interference principle (modified from Macleod, H. A. Thin-Film Optical Filters. Sciences New York 668 (Institute of Physics Publishing: 2001)). An incident ray of light (top left) traveling through a medium of refractive index n₁ at angle θ₁ will reflect off and also pass through at the interface of a new medium with higher refractive index n₂. Subsequently, a similar reflection will happen on the bottom end of the latter medium at angle θ₂. If the height of the second medium, d, is close to the wavelengths of the incident light, then constructive or destructive interference will take place, with the wavelength of greatest constructive interference (λ) occurring at integer multiples (m) given by the equation below at left. In the case of confocal microscopes, the incident light from the excitation lasers are essentially coming from directly above, so that θ₂=0, which collapses the equation as seen on the right. Here the incident ray is depicted in green, the reflection off the top of the higher n medium is depicted in red, and the reflection off the bottom is depicted in blue. (FIG. 13F), A schematic diagram demonstrating how the incident light in confocal microscope may be affected by the myelin surrounding an axon. The fibers that are being imaged herein are in aqueous environments, be it the cortex of the brain, the cerebrospinal fluid in the spinal cord, or phosphate buffer solution which bathes the sciatic nerve. The lipid rich myelin acts as the second higher refractive index medium, with the axon as another aqueous medium. It may be that the thickness of the myelin sheath acts generally together as a medium, wherein its thickness would be the determinant of which wavelengths constructively or destructively interfere (diagramed on the left). However, there may be more complex interactions, because myelin itself is layer upon layer of alternating lipids and proteins. Therefore the relative amount of protein, the density of lipids, and amount of residual cytosol in the myelin may be the determining factors for interference.

FIG. 14, comprising FIG. 14A and FIG. 14B, depicts a proposed model for the hour-glass shape of the reflected signal when visualized orthogonally. (FIG. 14A) Reconstructed orthogonal view of an SCoRe image of two myelinated axons showing the pattern of reflection (cyan) in comparison to the fluorescence of FM stained myelin (red) 2 μm scale bar. (FIG. 14B) Diagram of a possible explanation for the reflected signal from a myelinated axon having an hourglass shape in the orthogonal view and the reflection signal not intuitively appearing to come directly from the myelin.

FIG. 15 is a schematic of an exemplary system for detecting myelin.

FIG. 16 is a flow chart of an exemplary method for producing an image of the myelination at a region of interest.

### DETAILED DESCRIPTION

The present invention provides a system and method for detecting myelin and myelin-related disease. For example, the invention is based upon the finding that the combined reflectance of an administered multi-wavelength laser light specifically detects myelinated fibers. It is demonstrated herein that the present system and method effectively detects myelin, myelin defects, and myelin pathology. In certain embodiments, the invention is capable of imaging myelin deep within tissue without the need for high intensity laser power.

It is to be understood that the figures and descriptions of the present invention have been simplified to illustrate elements that are relevant for a clear understanding of the present invention, while eliminating, for the purpose of clarity, many other elements found in typical imaging systems. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention provides a system and method for the imaging of myelin and myelin defects. The present invention is based in part by the surprising discovery that spectral confocal reflectance microscopy (SCoRe) allows for high-resolution in vivo imaging of myelinated axons. For example, the present invention comprises the use of SCoRe for the imaging of myelinated axons in the intact brain, spinal cord and peripheral nerves. The invention allows for efficient identification and tracking of myelinated axons without the need for fluorescent labeling. Further, in certain embodiments, the invention provides for the detection of monitoring of myelin-related pathology.

In one embodiment, the invention allows for the generation of images using the principles of laser scanning confocal microscopy by the composite detection of simultaneously reflected signals from multiple wavelengths. It is demonstrated herein, that the system and method presented herein generates high signal-to-noise images specific to myelinated axons. In certain embodiments, the invention comprises the use of low laser power which, despite the low power, allows for the visualization of myelin deep within tissue. For example, in certain embodiments, the invention allows for imaging of myelin at a depth of up to 400 μm. In another embodiment, the invention allows for imaging of myelin at greater than 400 μm, by making use of longer laser wavelengths.

In certain embodiments, the invention comprises the administration of a multi-wavelength laser light to a sample, tissue, or subject of interest and collecting the reflected light of each of the wavelengths of the administered multi-wavelength light. As described herein, the combined reflectance of the wavelengths produces a complete image of myelinated axons. Further, the reflected spectra generate striking color patterns unique to individual myelinated fibers that allow their identification and tracing in dense axonal areas such as spinal cord and peripheral nerves. In certain embodiments, the invention allows for the identification of specific anatomical structures of myelinated axons including Nodes of Ranvier and Schmidt-Lanterman incisures. In certain embodiments, the reflectance pattern is highly sensitive to myelin alterations and can be used to detect the earliest changes in a variety of pathologies.

The system and method of the invention generates multicolor images based on SCoRe and takes advantage of the high refractive index of lipid-rich myelin (Fille and Peuker, 2000, J. Pathol. 190:635-8; Xiao et al., 2006, Brain Res. 1077:81-9). Reflection signals are obtained using a plurality of laser lines, which individually generate images of discontinuous segments but when merged, constitute a complete and sharp myelinated axon image.

As demonstrated herein, it is found that only myelinated sections of axons are reflective. This allows the clear identification in vivo of un-myelinated segments such as nodes of Ranvier. The merged reflection signals from the various lasers generate multicolor images of individual axons that have a unique overall color signature. This multicolor property allows the easy identification and tracing of individual processes in regions with high axonal density such as spinal cord and peripheral nerves. In addition, a unique reflection pattern is observed at Schmidt-Lanterman incisures allowing for their unambiguous identification in vivo. As such, the system and method of the present invention is very sensitive to myelin disruption, thereby allowing the present invention to detect myelin defects including, but not limited to peripheral nerve injury, demyelination, and developmental myelination defects. For example, in certain embodiments, the system and method of the invention can detect myelin damage in subjects having, or suspected to have myelin-related disorders, including but not limited to, multiple sclerosis, transverse myelitis, leukodystrophies, Guillain-Barre syndrome, diabetic neuropathy, inflammatory neuropathies ,inherited demyelinating conditions such as Charcot-Marie Tooth, Cancer related neuropathies, toxic neuropathies. In one embodiment, the system and method of the invention is used to characterize a tumor. For example, in one embodiment, the method is used to diagnose a tumor as being produced from or caused by myelin producing cells. In another embodiment, the method is used to determine the aggressiveness or potential for growth of a tumor. For example, the method is used to detect the presence and extend of myelinated fibers at or near a tumor site, which is associated with tumor aggressiveness.

In certain embodiments, the system and method of the invention provides short-term and long-term time lapse imaging of myelinated axons, or myelin defects, in a sample, tissue, or subject of interest. In certain embodiments, the invention provides for deep tissue imaging using laser intensities much lower than those needed for fluorescence imaging, thus minimizing photobleaching and phototoxicity. In certain embodiments, the method comprises the use of near-infrared wavelengths which allow for high-resolution in vivo deep tissue imaging of myelinated fibers and myelin defects.

In one embodiment, the system and method of the invention comprises imaging an underlying region of interest. For example, in certain embodiments, imaging of a region of interest can comprise imaging through a cranial window, pial layer, skin, muscle, fat, dura matter, eye, and the like. That is, the present invention provides for deep tissue imaging, such that, in certain instances, the imaging system of the invention need not be directly inserted to the region of interest. In certain embodiments, the imaging system of the invention can be advanced into the sample, tissue, or subject. For example, in certain embodiments, the system can be advanced through a catheter, into the vicinity of the region of interest within a subject.

In one embodiment, the system and method of the invention comprises simultaneous SCoRe imaging, as described herein, with confocal fluorescence microscopy. In certain instances, the combination of imaging modalities allows for the identification of interactions between myelinated axons and adjacent astrocytes and microglia.

### System

In one embodiment, the present invention provides a system for detecting myelinated fibers, myelin defects, or myelin-associated pathology. As described elsewhere herein, the system of the invention is based upon SCoRe imaging by administering to a sample a multi-wavelength laser light and collecting the reflected light of each wavelength, which when the reflectance of the wavelengths are combined produces a complete image of a myelinated axon or fiber.

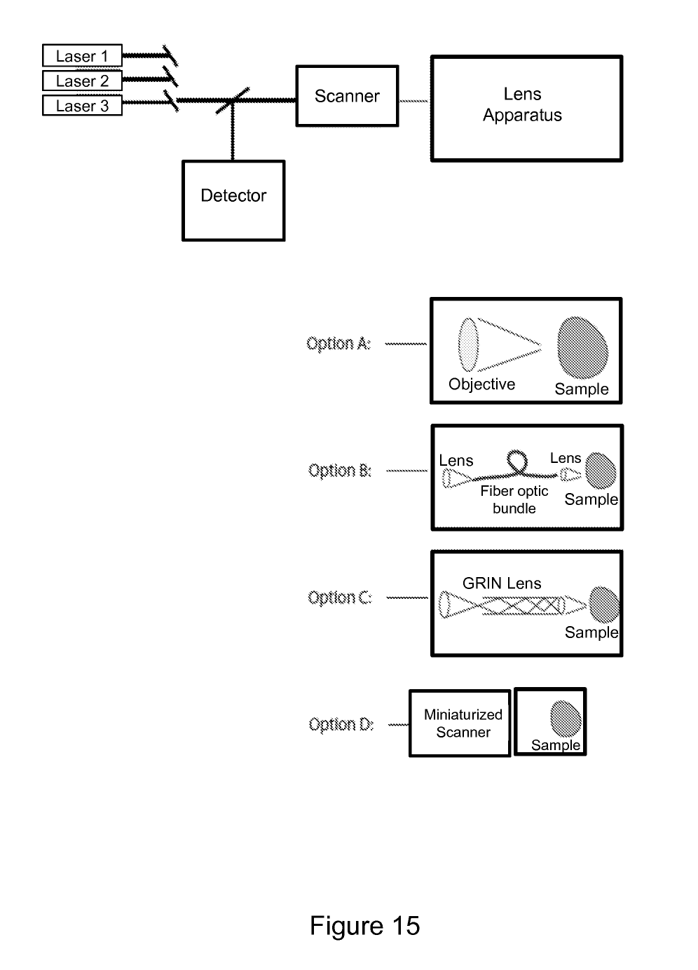

FIG. 15 depicts an exemplary system of the present invention. The system comprises a one or more lasers, wherein the one or more lasers collectively produce a plurality of beams of laser light, each having a different wavelength. By plurality of beams, it is meant that in certain embodiments, the system produces 2, 3, 4, 5, 10, or more different beams. In one embodiment, the system comprises a plurality of lasers, wherein each of the plurality of lasers emits light at a different wavelength. In another embodiment, the system comprises one or more lasers, wherein at least one of the one or more lasers is capable of producing multiple different wavelengths. In one embodiment, the system is capable of simultaneously administering all of the plurality of beams, thereby administering a multi-wavelength laser light to the sample.

In certain embodiments, the system makes use of three different wavelengths. For example in one embodiment, the system comprises a 488 nm laser, a 561 nm laser, and a 633 nm laser. In one embodiment, the system comprises a 488 nanometer (nm) output of an Argon laser, a 561 nm output of a Diode Pumped Solid State (DPSS) laser, and a 633 nm output from a Helium/Neon laser. However, the present invention is not limited to any particular wavelength, or combinations of wavelengths. Rather, other wavelength combinations could be used. In one embodiment, each wavelength is at least about 20 nm apart from each other. In one embodiment, each wavelength is at least about 70-100 nm apart. The wavelengths of the multi-wavelength laser light may be chosen from the range of about 400 nm to about 1300 nm

In one embodiment the system comprises a supercontinuum (white-light) laser, wherein the plurality of beams can be each generated by the single laser. For example, the wavelengths of the plurality of beams could be chosen anywhere in the laser's available spectrum (about 470-670 nm).

In another embodiment, the system comprises a pulsed Ti-Sapphire laser, where laser light can be sent through multiple optical parametric oscillators (OPOs) to produce a plurality of beams in the infrared spectrum (1000-1300 nm). In certain embodiments, a system comprising infrared wavelengths would have the advantage of deeper penetration through scattering tissue (e.g. bone).

As described elsewhere herein, in certain embodiments, the invention necessitates the use of only low intensity laser power, which still allows for imaging deep within a sample or tissue of interest. For example in certain embodiments, the intensity of each laser is about 200-400 microwatts. Therefore, in certain embodiments, the system comprises hand-held, battery-operated lasers, which while having much less power compared to standard lasers used for confocal microscopy, would still be able to produce informative images of myelin fibers and defects. For example, in one embodiment, the system comprises one or more laser diodes, which in certain instances are less expensive and portable than other lasers.

In one embodiment, the plurality of beams of laser light are combined to produce a multi-wavelength laser light before entering a scanner. The scanner of the system may comprise any suitable confocal scanning unit or confocal scanner known in the art. For example in one embodiment, the scanner of the system comprises a set of galvanometer-controlled mirrors. In one embodiment, the combined light is raster scanned by the galvanometer-controlled mirrors of the scanner.

However, the present invention is not limited to the use of a laser scanning confocal system. Rather, in certain embodiments, the system comprises a “spinning disc” based confocal microscopy system (Nipkow disk), which allows for rapid confocal imaging based upon the Yokagawa confocal.

In another embodiment, the system of the invention utilizes reflectance microscopy without the need for confocal imaging. For example, in certain embodiments, the system of the invention uses post-processing algorithms, including, for example, deconvolution, to produce high quality images without the need for a confocal-based imaging system.

In one embodiment, the combined light is passed through a high numerical objective lens, out of the system, and onto the sample, tissue, or subject of interest.

The reflected light of sample, tissue, or subject of interest, then propagates back through the objective lens and into the scanner, wherein the reflected light is descanned by the mirrors contained therein. In one embodiment, the reflected light is split by a prism so that each wavelength of the administered multi-wavelength beam can be detected separately by a plurality of photodetectors, a single photodetector for each wavelength. In certain embodiments, the system comprises one or more photodetectors capable of detecting wavelengths in the infrared spectrum. The photodetectors may be any suitable photodetector, including photomultiplier (PMT) detectors.

In another embodiment, the system emits each of the plurality of beams one at a time, thereby allowing for the collection and detection of the reflected light of each wavelength one at a time. For example, in certain embodiments, the system cycles through the plurality of beams individually, rather than simultaneously imaging all of the wavelengths.

The system of the invention comprises a lens apparatus for guiding the multi-wavelength laser light to the sample and for collecting the reflected light from the sample. As depicted in FIG. 15, the system of the invention encompasses a variety of embodiments that can be used to deliver and collecting light.

In one embodiment, as depicted in option A, the system comprises an objective lens that makes direct contact with a sample or tissue of interest. For example, in one embodiment, the system comprises a long working distance water immersion objective lens.

In one embodiment, as depicted in option B, the system comprises a fiberoptic bundle extension with a microobjective lens positioned at the tip of the bundle. For example, in certain embodiments, the system comprises a microprobe or needle objective lens. An exemplary microprobe lens includes the microprobe objectives distributed by Olympus (Center Valley, Pa.). This allows for minimally invasive access to small and remote areas such as peripheral nerves, subarachnoid space, pial surface, and the like.

In one embodiment, as depicted in option C, the system of the invention comprises Gradient-index (GRIN) optics. GRIN optics, in certain instances, is used to reduce spherical aberrations and improve resolution and allow deep tissue imaging with thin needle-like probes which can be used to provide minimally invasive access to areas such as the subarachnoid space, pial surface, and the like.

In one embodiment, as depicted in option D, the system comprises a miniaturized motorized scanner that uses Lissajous patterns of motion for rapid and multipositional scanning. This is useful for in vivo imaging to reduce the artifactual effect of motion. These types of scanners can be directly attached to the subject due to their small size.

In certain embodiments, one or more components of the lens apparatus are housed such that the lens may be advanced to a tissue of interest in vivo. For example, in certain embodiments, the lens apparatus may be advanced through a catheter or other hollow access tube.

In certain embodiments, the system of the invention further comprises one or more additional components that are coupled to, or can be used in conjunction with, the imaging system components described herein. For example, in certain embodiments, the system comprises tissue processing equipment, experimental instrumentation, and the like. For example, in one embodiment, the system comprises any tissue sectioning device known in the art, including, but not limited to a microtome. In one embodiment, the system comprises electrophysiology instrumentation for recording or measuring signals from the imaged tissue. The system may further comprise any equipment used during experimentation, such as environmentally controlled chambers, fluidic delivery devices, and the like.

In one embodiment, the system of the invention comprises a computing device. In one embodiment, the computing device controls the laser or lasers of the system, providing user control of laser intensity, wavelength, and the like. In one embodiment, the computing device receives information regarding the detected reflected light in order to construct single-wavelength and combinatorial multi-wavelength images.

The computing device may include a desktop computer, laptop computer, tablet, smartphone, watch, televisions, or other device and include a software application platform or portal providing a user interface as contemplated herein. In certain embodiments, the computing device is network enabled. The applications platform may be a local or remotely executable software platform, or a hosted internet or network program or portal. The computing devices may include at least one processor, standard input and output devices, as well as all hardware and software typically found on computing devices for storing data and running programs, and for sending and receiving data over a network. The communications network between the computing device and the vibrator component can be a wide area network and may be any suitable networked system understood by those having ordinary skill in the art, such as, for example, an open, wide area network (e.g., the internet), an electronic network, an optical network, a wireless network, personal area networks such as Bluetooth, a physically secure network or virtual private network, and any combinations thereof. In certain embodiments, wireless communication may be via a wide area network and may form part of any suitable networked system understood by those having ordinary skill in the art for communication of data to additional computing devices, such as, for example, an open, wide area network (e.g., the internet), an electronic network, an optical network, a wireless network, a physically secure network or virtual private network, and any combinations thereof. Such an expanded network may also include any intermediate nodes, such as gateways, routers, bridges, internet service provider networks, public-switched telephone networks, proxy servers, firewalls, and the like, such that the network may be suitable for the transmission of information items and other data throughout the system.

Data transfer can be made via any wireless communication may include any wireless based technology, including, but not limited to radio signals, near field communication systems, hypersonic signal, infrared systems, cellular signals, GSM, and the like. In some embodiments, data transfer is conducted without the use of a specific network. Rather, in certain embodiments, data is directly transferred to and from the system components.

As would be understood by those skilled in the art, the system components, including the computing device, may be wirelessly connected to the expanded network through, for example, a wireless modem, wireless router, wireless bridge, and the like. Additionally, the software platform of the system may utilize any conventional operating platform or combination of platforms (Windows, Mac OS, Unix, Linux, Android, etc.) and may utilize any conventional networking and communications software as would be understood by those skilled in the art.

To protect data, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. Access or use restrictions may be implemented for users at any level. Such restrictions may include, for example, the assignment of user names and passwords that allow the use of the present invention, or the selection of one or more data types that the subservient user is allowed to view or manipulate.

The computing device may include a user interface including a display screen to provide text or other graphics indicating user or system information. The user interface may also include one or more depressible buttons, dials, recessed switches or a touch screen through which the system may be programmed or controlled by a user.

The software may include a software framework or architecture that optimizes ease of use of at least one existing software platform, and that may also extend the capabilities of at least one existing software platform. The software provides applications accessible to one or more users to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system. Presentation of data through the software may be in any sort and number of selectable formats. For example, a multi-layer format may be used, wherein additional information is available by viewing successively lower layers of presented information. Such layers may be made available by the use of drop down menus, tabbed pseudo manila folder files, or other layering techniques understood by those skilled in the art.

The software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text or phone call, to alert a user.

In one embodiment, the computing device of the system comprises a specialized software platform to construct images of myelinated fibers using the combination of reflected light, as described herein. The software may be used to track individual fibers over time. In one embodiment, the software may automatically notify the user of anatomical features, including Nodes of Ranvier and Schmidt-Lanterman incisures.

In one embodiment, the system of the invention is configured for hand-held or portable use to allow for effective imaging in clinical environments. For example, as described herein, in certain embodiments, the laser or lasers of the system are low powered hand held lasers, which still produce enough intensity to provide for deep tissue myelin imaging.

### Method

The present invention includes a method of detecting and monitoring myelinated fibers and myelin defects. For example, it is described herein that SCoRe can be used to effectively detect myelinated axons. It is demonstrated that the reflectance of multiple wavelengths allows for the specific detection of axons or fibers that are myelinated. For example, the combination of the collected reflected light of a multitude of wavelengths (or “colors”) leads to a more complete image of myelin. Further, it is demonstrated that the method is able to detect specific anatomical features of myelinated fibers.

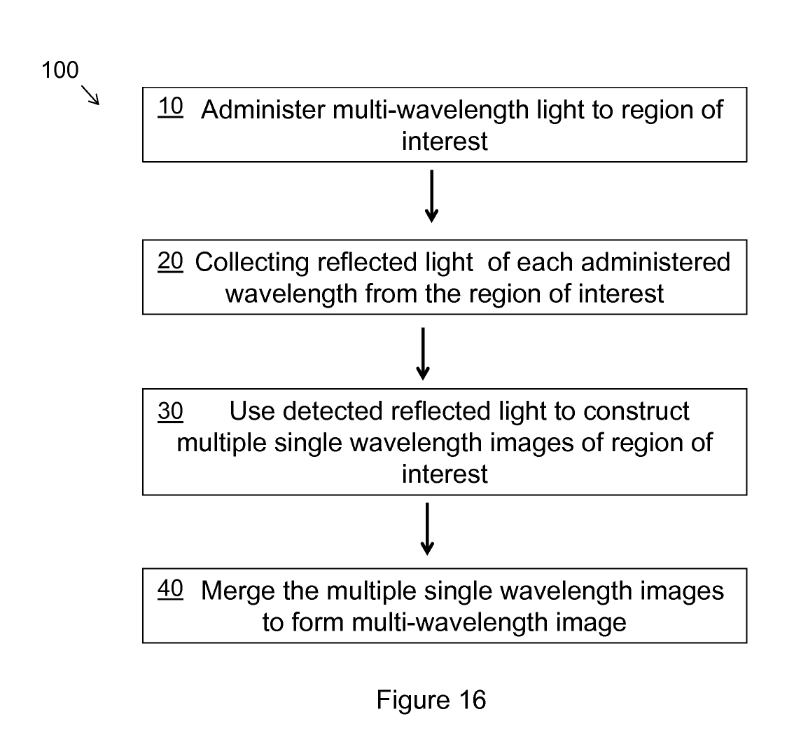

FIG. 16 depicts an exemplary method 100 of the present invention for forming an image of myelination of a region of interest. In one embodiment, method 100 comprises administering multi-wavelength light to the region of interest (10). For example, in certain embodiments, administering (10) comprises irradiating the region with multiple single wavelength beams, each having a different discrete wavelength. In one embodiment, method 100 comprises collecting the reflected light of each administered wavelength (20). For example, the collected light from the region may be filtered and/or split into narrow bands centered at about each administered wavelength. Method 100 further comprises using the detected reflected light to construct multiple single wavelength images of the region (30). Each of the single wavelength images can then be merged to form a multi-wavelength image (40). In one embodiment, the multi-wavelength image can be a single-color image, where all wavelengths are represented by a single color thereby providing a complete image of myelination at the region. In one embodiment, the multi-wavelength image is a multi-color image, where each single-wavelength is represented by a different color.

The method of the invention comprises administering multi-wavelength laser light to a region of interest. For example, in certain embodiments, the method comprises administering multi-wavelength laser light having 2, 3, 4, 5, 10, or more different wavelengths to the region. As described elsewhere herein, the plurality of wavelengths may arise from one or more different laser lines. In certain embodiments, the method comprises combining a plurality of single-wavelength beams into a multi-wavelength laser light. In one embodiment, the method comprises administering 488 nm, 561 nm, and 633 nm wavelengths to the region. However, the present invention is not limited to the precise number, types, or combinations of wavelengths used. In one embodiment, each wavelength is at least about 20 nm away from each other. In another embodiment, each wavelength is at least about 70-100 nm away from each other.

In certain embodiments, the laser intensities of the administered multi-wavelength laser light is about 200-400 μW at the sample. The present method allows for such low-intensity, while still providing effective imaging deep within tissue. For example, in certain embodiments, the method provides for imaging at depths of up to about 400 μm. In another embodiment, the method provides for imaging at depths of greater than 400 μm. The precise intensity of the light may be adjusted depending on the type of sample or tissue being imaged, the depth of the region of interest, and the length of the wavelengths used.

In one embodiment, the method comprises passing the laser light through a filter, for example an Acousto-Optical Tunable Filter (AOTF), prior to reaching the region of interest. In one embodiment, the method comprises passing the laser light through a mirror, for example a 30/70 partially reflective mirror, prior to reaching the region of interest. It would be understood by those skilled in the art that one or more of the wavelengths of the administered laser light may be filtered, reflected, or otherwise altered using any standard imaging components known in the art.

In certain embodiments, the method comprises passing the laser light through a high numerical objective lens prior to reaching the subject. The objective lens may be any standard or known lens known in the art. The precise lens used may be dependent upon the type of sample or region being investigated as well as the imaging needs of the application. For example, the magnification of the objective lens may be chosen as necessary for the given application.

In one embodiment, the method comprises collecting reflected light from the sample being imaged. For example, in certain embodiments, light collected from the sample is collected through narrow bands, for example using a prism and mirror-sliders, to split the reflected light into the wavelengths of the administered multi-wavelength laser light. For example, in one embodiment, the reflected light is collected through narrow bands centered around 486-491 nm, 559-564 nm, and 631-636 nm respectively.

In one embodiment, the method comprises detecting the reflected light at each wavelength. For example, in one embodiment, photodetectors are used to detect the reflected light from each wavelength. The photodetectors may be any suitable photodetector, including photomultiplier (PMT) detectors.

The method comprises using the detected reflected light at each wavelength to construct an image which depicts myelin at the imaged region. In one embodiment, the light detected at each photodetector is considered independently to construct a single-wavelength image for each of the wavelengths. In one embodiment, the single-wavelength images are combined or merged to form a multi-wavelength image. That is, in certain embodiments, the multi-wavelength image is a merged image of all reflected wavelengths. In one embodiment, a multi-color image is constructed, wherein each reflected wavelength is depicted in an individual color. For example, a plurality of single color images, each generated based upon each individual wavelength, may be then combined into a merged or multi-color image. In another embodiment, the multi-color image may be pseudo-colored to provide a single-color image that represents the combined reflectance of all wavelengths. As described herein, in certain embodiments, a multi-color image provides a unique pattern of each individual myelinated fiber, thereby allowing for the tracking of that particular fiber. Images may be constructed or analyzed using any standard hardware and software platforms known in the art.

The method of the invention can be performed on any subject, including, for example, a human subject. In certain embodiments, the method comprises the imaging of myelin in a tissue sample, for example either a fixed or non-fixed sample. The sample may be any suitable sample obtained from a subject, including, for example, a biopsy sample or post-mortem sample. The sample may be of any suitable tissue including, but not limited to, spinal cord, peripheral nerves, brain, muscle, skin, and the like.

In certain embodiments, the method of the invention comprises the in vivo imaging of myelin in a subject of interest. For example, the subject may be a human, or other mammal. In certain embodiments, the subject is a patient being evaluated for the presence of a myelin-related disorder. In another embodiment, the subject is a research subject, including but not limited to, a mouse, rat, hamster, guinea pig, cat, dog, monkey, or the like, being studied. For example, the method of the invention may be used to examine the effect of a given stimulus (e.g., treatment, injury, etc.) on the myelin structure or composition of the research subject.

In certain embodiments, the method comprises non-invasive or minimally invasive in vivo imaging of myelin. For example, in certain embodiments, the method comprises imaging of myelin deep within tissue, thereby allowing for imaging to be conducted from outside the body of the subject. For instance, the present method allows for deeper imaging through scattering tissue compared to confocal fluorescent microscopy. Therefore, in certain embodiments, imaging of the region of interest can be done through the skin or eye of the subject.

In another embodiment, the method comprises advancing one or more imaging components of the invention towards a region of interest. For example, in certain embodiments, the method comprises advancing a scope (e.g., endoscope), or other imaging device, capable of administration of multi-wavelength laser light, to the region of interest. In certain embodiments, the scope can be advanced into or in the vicinity of the brain, spinal cord, or peripheral nerves.

In one embodiment, the method of the invention is used to evaluate the status of myelin or myelinated axons in a subject. For example, the method may be used to diagnose a subject as having a myelin-related or myelin-associated disease. In another embodiment, the method may be used to monitor the effectiveness of a treatment or therapy of a myelin-related disease in a subject. For example, the method may be used to determine if a treatment or therapy is promoting the re-myelination of a fiber or is reducing the amount of de-myelination.

Exemplary myelin-related diseases or disorders that can be detected, diagnosed, or monitored using the present invention include, but are not limited to multiple sclerosis, transverse myelitis, leukodystrophies, Guillain-Barre syndrome, diabetic neuropathy, inflammatory neuropathies ,inherited demyelinating conditions such as Charcot-Marie Tooth, Cancer related neuropathies, and toxic neuropathies.

In one embodiment, the method of the invention provides for the detection of tumors produced by myelin producing cells. For example, the present invention can be used for the detection of schwannomas, neurofibromas, malignant peripheral nerve sheath tumors, oligodendrogliomas, and the like. In certain embodiments, the method detects the presence or extent of myelin in a tumor to classify the tumor as being formed of or caused by myelin producing cells. For example, the present method can detect unique reflective properties of myelin within tumors in order to classify the tumor as being formed or caused by myelin producing cells. Thus, the method provides a diagnosis of schwannoma, neurofibroma, malignant schwannoma, oligodendroglioma, and the like, by detecting the presence or extent of myelin within a tumor.

In one embodiment, the method of the invention provides for the characterization of a tumor. For example, the growth or aggressiveness of a cancerous tumor may be determined by evaluating the presence of myelinated fibers in and around the tumor. The method comprises imaging myelin at the tumor site, for example by in vivo imaging by advancing a scope or probe of a system capable of SCoRe imaging to the tumor site. In another embodiment, the presence of myelin at the tumor site is evaluated by SCoRe imaging of an excised or biopsied tissue sample. The characterization of any benign tumor or tumor associated with cancer, for example prostate cancer, by detecting the presence and extent of myelinated fibers at the tumor site may be performed using the present method.

In one embodiment, the method comprises a method of early diagnosis of multiple sclerosis. For example, in certain embodiments, the method comprises imaging of myelin with the brain of a patient being evaluated for multiple sclerosis. The present method demonstrates the ability to look for myelin damage, and thus the present method could detect subtle damage prior to the ability to detect myelin damage using MRI.

In one embodiment, the method of the invention provides for the detection of Nodes of Ranvier in myelinated axons. For example, it is demonstrated herein that Nodes of Ranvier appear as small gaps in the combined reflected image.

In one embodiment, the method of the invention provides for the detection of Schmidt-Lanterman incisures. It is demonstrated herein that Schmidt-Lanterman incisures appear as lines of alternating colors orthogonal to the direction of the myelin fiber.

In one embodiment, the method of the invention produces images where individual myelinated fibers within nerve bundles have a color “signature” which allows them to be tracked by their unique color pattern. It is demonstrated herein that this unique color pattern is disrupted when the myelin structure is damaged.

In one embodiment, the method comprises the imaging of myelin during surgery or other clinical procedure in order to effectively warn a clinician of the presence and location of myelinated fibers. This would allow the clinician to at least attempt to leave the myelinated fibers unharmed through the procedure. For example, in one embodiment, the method comprises imaging of myelin during the resection of a tumor located in the head, neck, brain, spinal cord, or peripheral nerve. The method could be used to detect myelinated white matter near the tumor to avoid unnecessary harm to the myelinated tracts.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Label-Less Multicolor In Vivo Imaging of Myelinated Axons in Health and Disease with Spectral Confocal Reflectance Microscopy

Presented herein is a new technique based on spectral confocal reflectance microscopy (SCoRe) for high-resolution in vivo imaging of myelinated axons in the intact brain, spinal cord and peripheral nerve that requires no fluorescent cellular labeling. Images are generated with a conventional laser scanning confocal microscope by the composite detection of simultaneously reflected signals from multiple lasers of various wavelengths. This generates high signal-to-noise images specific to myelinated axons that with low laser power can reach depths up to 400 μm or potentially greater with longer laser wavelengths. The reflected spectra generate striking color patterns unique to individual myelinated fibers that allow their identification and tracing in dense axonal areas such as spinal cord and peripheral nerves and can highlight nodes of Ranvier and Schmidt-Lanterman incisures. Furthermore, this pattern is highly sensitive to myelin alterations and can be used to detect the earliest changes in a variety of pathologies. Using SCoRe chronic high-resolution transcranial imaging was performed in mice, capturing for the first time de novo myelination of cortical axons in vivo. Furthermore, the feasibility of imaging myelinated axons in the human cerebral cortex through the pial layer is demonstrated herein. SCoRe adds a powerful component to the evolving imaging toolbox to study myelination in living animals and potentially in humans.

The materials and methods employed in these experiments are now described.

### Animals

Mouse lines used included Thy1-YFP line H (Feng et al., 2000, Neuron 28:41-51) (Jackson Labs # 003782), CX3CR1-GFP (Jung et al., 2000, Mol. Cell Biol. 20:4106-4114) (Jackson Labs #005582), MBPshi (Jackson Labs #001428), mT/mG (Jackson Labs #007576) and C57BL/6 (Jackson Labs #000664).

### Spectral Confocal Reflectance Microscopy

A Leica SP5 confocal microscope with a 488, 561, and 633 nm laser wavelength outputs sent through an Acousto-Optical Tunable Filter (AOTF) and a 30/70 partially reflective mirror was used. The reflected light was collected using three PMTs set to collect light through narrow bands defined by prism and mirror-sliders, centered around the laser wavelengths, 486-491 nm, 559-564 nm, and 631-636 nm respectively. The channels from each PMT were then considered independently, additively combined into one channel, or shown as a color composite with 488 as blue, 561 as green, and 633 as red. Laser intensities for SCoRe ranged from 200-400 μW at the sample depending on the preparation used (thin skull vs. cranial window), the tissue being imaged, and the specific laser as longer wavelength lasers required less power for sufficient signal and penetration. Images were analyzed using NIH ImageJ. 3D reconstructions were created with Imaris imaging software (Bitplane Scientific Software).

### In vivo Imaging of the Mouse Cortex

The thin skull procedure was used for acute and chronic transcranial imaging as described previously (Grutzendler et al., 2002, Nature 420:812-6) while the cranial window procedure was used for acute imaging sessions with dye labeling. Briefly mice were fully anesthetized using isoflurane (MBP shiverer) or Ketamine Xylazine, and the scalp was shaved and sterilized. A midline scalp incision was made, and a custom made metal plate was affixed to the skull using cyanoacrylate. An area no more than 1 mm was thinned with a high speed drill and a microsurgical blade to a thickness of 20-30 μm for the thin skull preparation, or removed along with the underlying dura for the cranial window. For fluorescent myelin labeling, fluoromyelin (Life Technologies) was applied directly to the exposed cortex in a 50% dilution in PBS from stock solution for 45 minutes and then washed thoroughly. Occasionally we observed reflective fibers that were not labeled with fluoromyelin which was likely due to regional variation in dye penetration resulting in incomplete dye labeling. For astrocyte labeling 50 μM sulforhodamine 101 (Grutzendler et al., 2002, Nature 420:812-6) dissolved in PBS was applied for 20 minutes to the exposed cortex and then washed thoroughly. A #0 glass coverslip cut to size was placed over the cranial window and glued in place using cyanoacrylate.

### In Vivo Imaging of the Sciatic Nerve

Mice were fully anesthetized using Ketamine Xylazine and the skin was thoroughly shaved and sterilized on the lower back and upper thigh. A small incision was made in the skin above the plane between the vastus lateralis and the biceps femoris muscles. The skin was gently dissected from the underlying musculature and the sciatic nerve was exposed by separating the vastus lateralis and biceps femoris and separated from the surrounding connective tissue. A custom made metal rod was used to gently elevate the separated nerve to immobilize it for imaging. After imaging the nerve was lowered back into its original location and the incision was sutured. In some cases fine #5 forceps were used to cause a controlled crush injury of the nerve. The nerve was pinched for 20 seconds to cause a complete reproducible crush without severing the nerve. The nerve was imaged before, immediately after, and at one subsequent time point (5-10 days). Otherwise, for acute myelin damage, 1 μL of 2.5% lysophosphatidylcholine (LPC, Sigma Aldrich), 100% DMSO, or 100% double distilled water was injected into the sciatic nerve with a small pulled glass pipette. Additionally myelin within the sciatic nerve was sometimes labeled with a direct injection of 0.5-2 μL of Fluoromyelin using a glass electrode. This was necessary because the dyes would not dissolve across the surrounding epineurium.

### In Vivo Imaging of the Spinal Cord

Mice were fully anesthetized using Ketamine Xylazine and the back of the mouse was shaved and sterilized with alcohol and betadine. A dorsal midline incision of 1.5 cm was made over vertebrae T10 to L2 and the muscles surrounding the spinous and transverse processes were gently removed to expose the underlying vertebrae. Two standard sterilized staples were used as small anchors and were glued with surgical grade cyanoacrylate glue to the pedicles of the vertebrae then further secured with dental cement (Fenrich et al., 2012, J. Physiol. 590:3665-75). A small custom shaped metal rod was placed in the dental cement to serve as an anchor point to immobilize the animal during imaging sessions. Next a laminectomy was performed on 1 vertebra using a small set of dissecting scissors. 1% low melting agarose was applied to the exposed spinal cord and then a #0 glass coverslip was secured on top of the agarose with cyanoacrylate and then dental cement. After the dental cement had dried the animal was secured to a custom built holder for imaging. In some cases 1 μL of fluoromyelin dye was injected into the spinal cord to label myelin.

### Human Explant Imaging

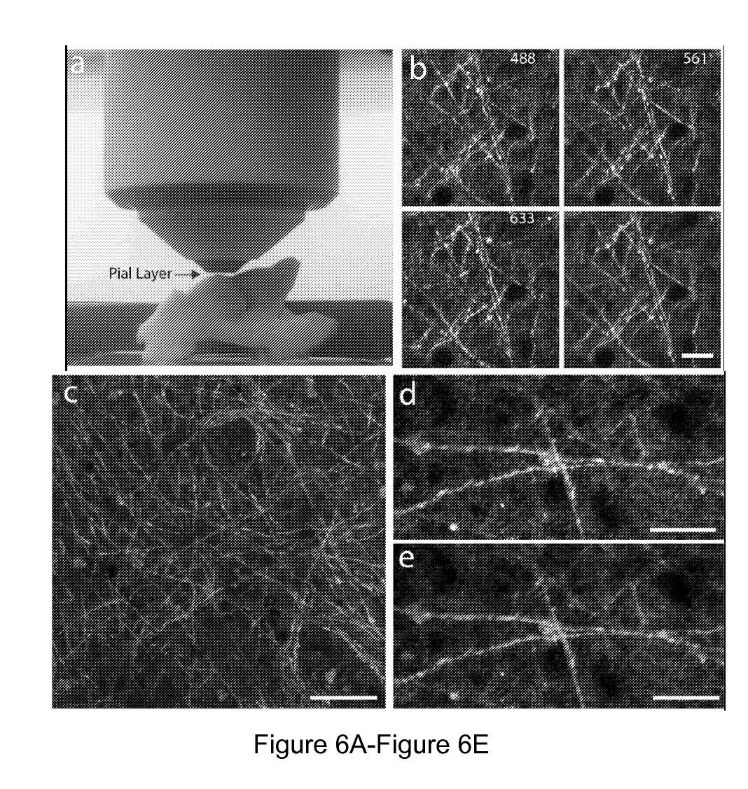

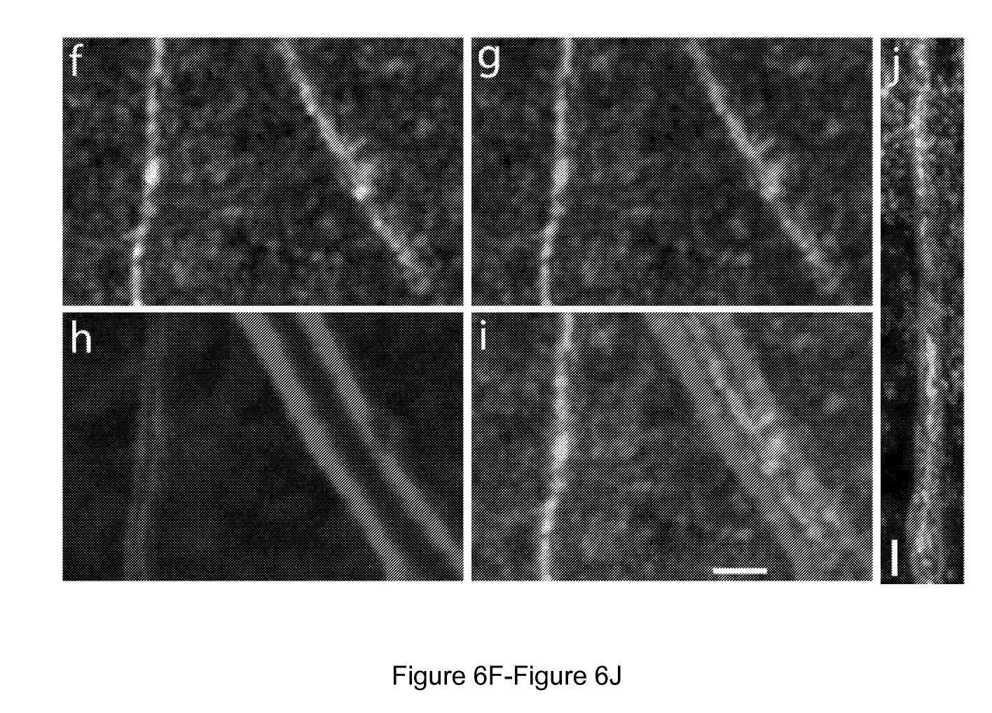

A 4% paraformaldehyde fixed explant of human tissue from the entorhinal cortex was oriented to perform SCoRe imaging through the pial surface as shown in FIG. 6a. Laser intensities necessary for optimal SCoRe signal were similar to those used for in vivo mouse imaging (200-400 μW at the sample) with a 20×1.0 NA water immersion objective. Fluoromyelin was applied to the cortical surface for 20 minutes to label myelin within the superficial cortex and then washed thoroughly with PBS.

The results of the experiments are now described.

### Spectral Confocal Reflectance Microscopy (SCoRe) Enables Visualization of Myelinated Axons in Vivo

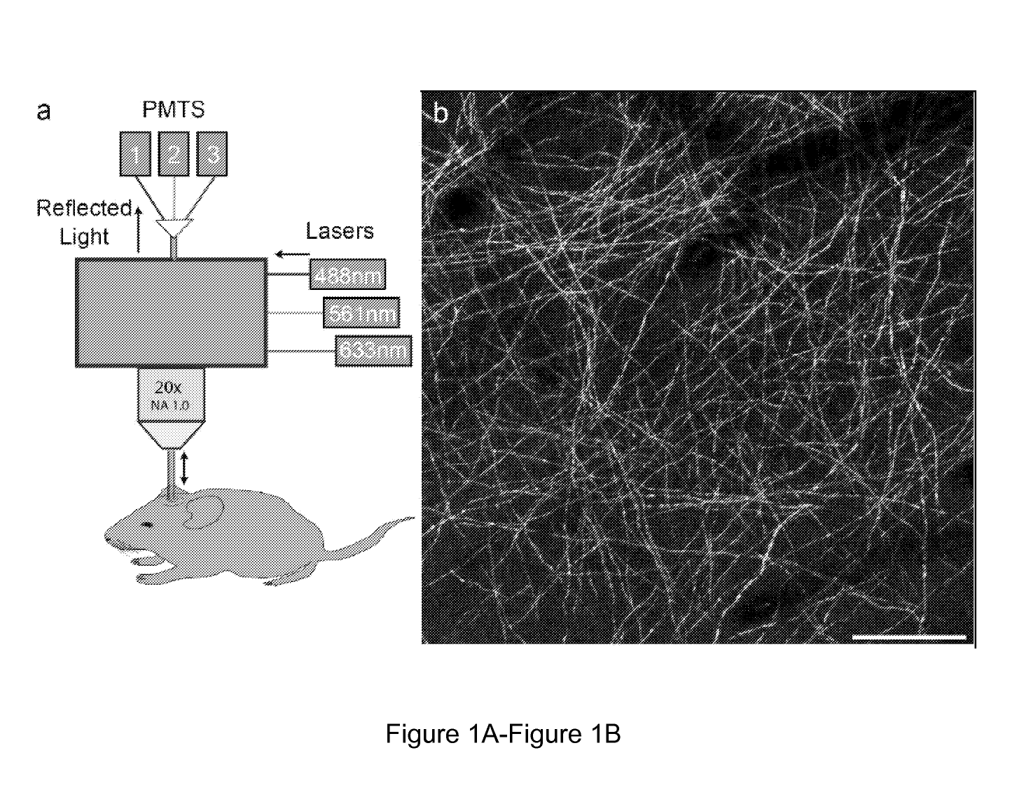

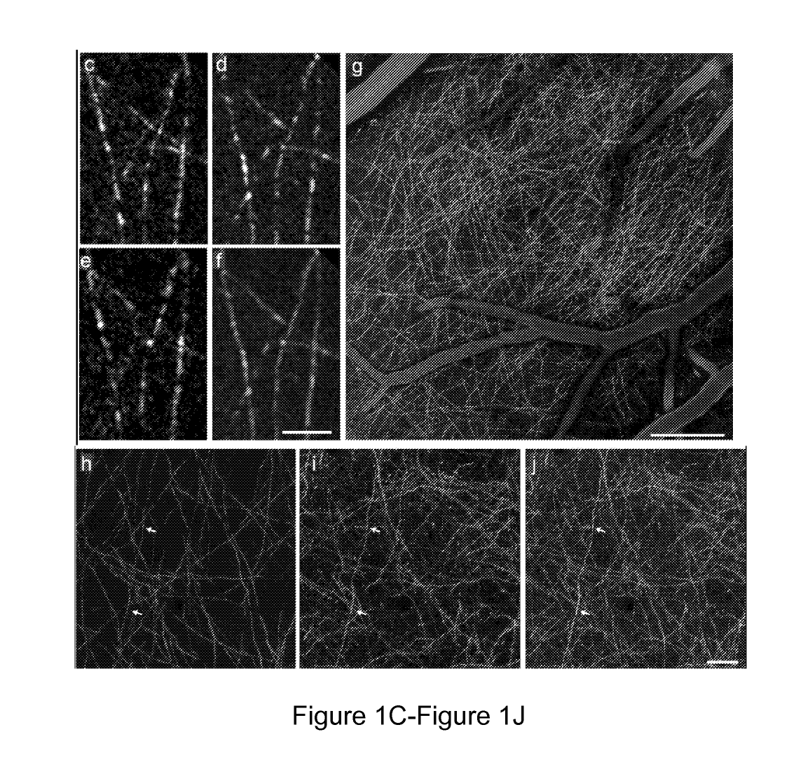

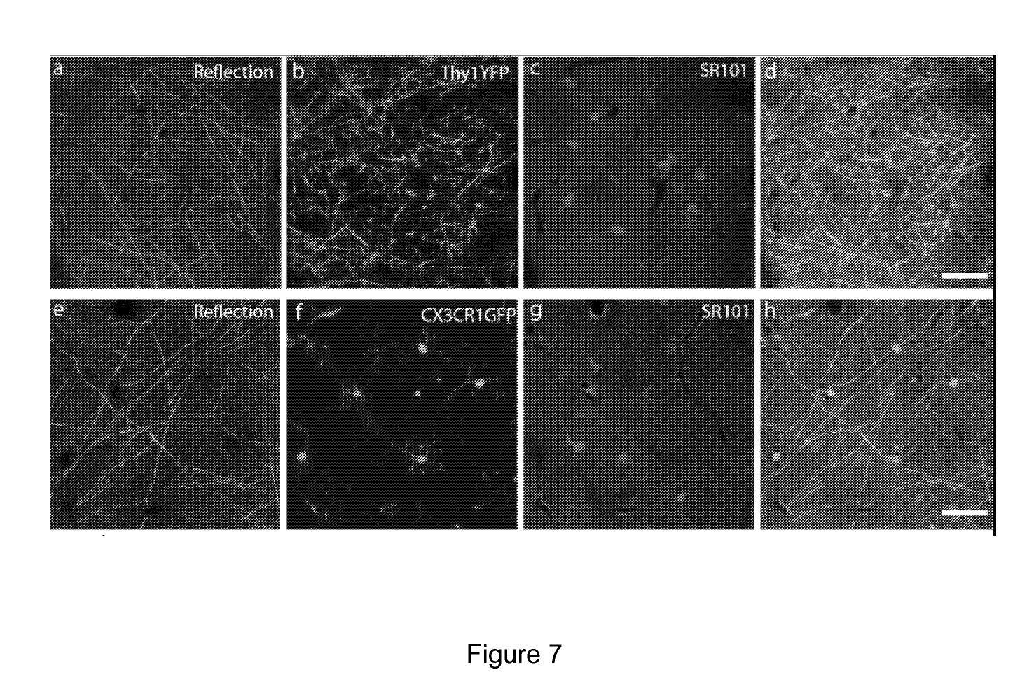

Single-wavelength confocal reflection microscopy was first applied through a thin skull window preparation in an anesthetized mouse. Surprisingly, a peculiar reflective fiber network pattern was observed below the skull and meninges. Although this pattern was patchy and incomplete (FIG. 1C), its location and distribution was reminiscent of cortical layer I axons as visualized by in vivo two photon fluorescence microscopy (Grutzendler et al., 2002, Nature 420:812-6). It was then noticed that when using different laser wavelengths, the images remained patchy but the patterns were nonoverlapping for each wavelength (FIG. 1C-FIG. 1E). Interestingly, simultaneous imaging with various laser wavelengths (488, 561, 633 nm) gave a complementary reflection pattern that when composited (FIG. 1F) and pesudocolored uniformly (FIG. 1G, cyan), appeared as a continuous image revealing that these processes projected for long distances with relatively moderate density. To better characterize the cellular nature of these reflective processes, reflectance imaging was able to be combined with in vivo confocal microscopy in transgenic mice expressing fluorescent proteins or after labeling with dyes (FIG. 1G-FIG. 1J and FIG. 7).

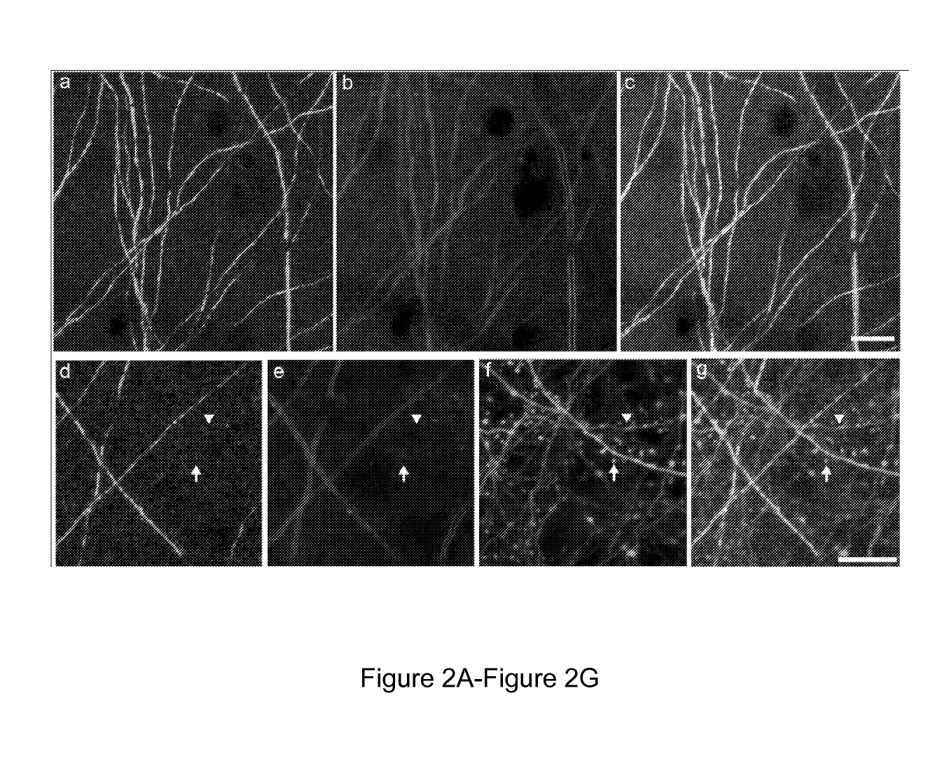

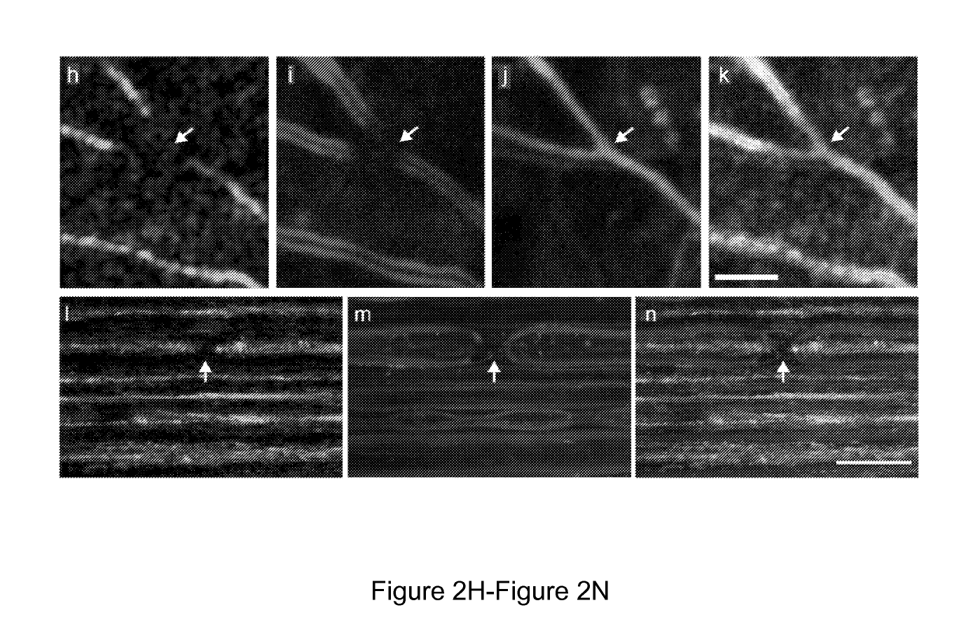

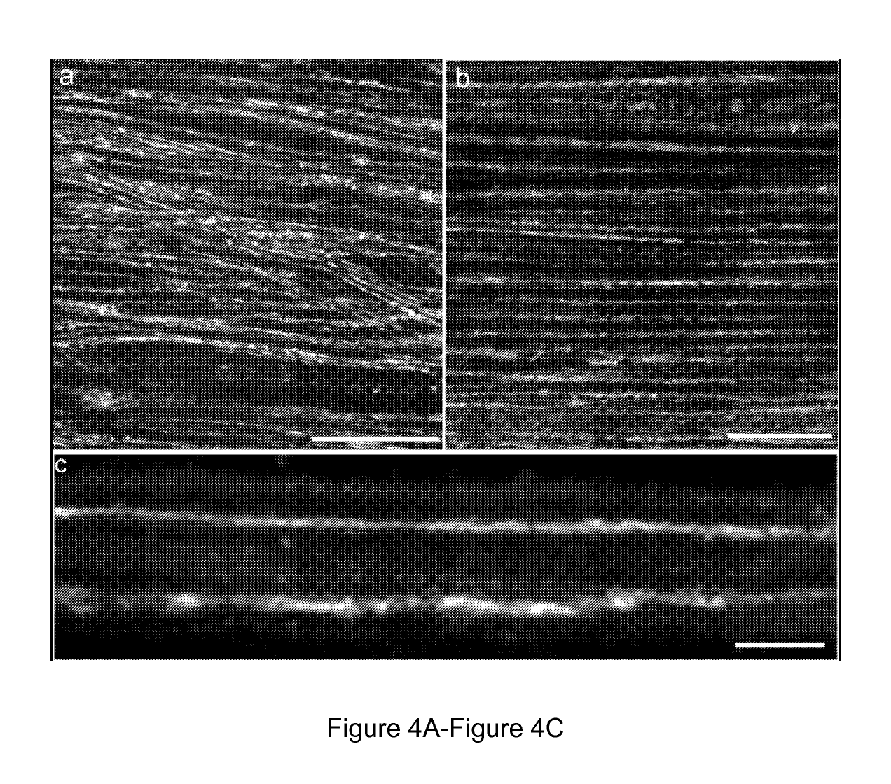

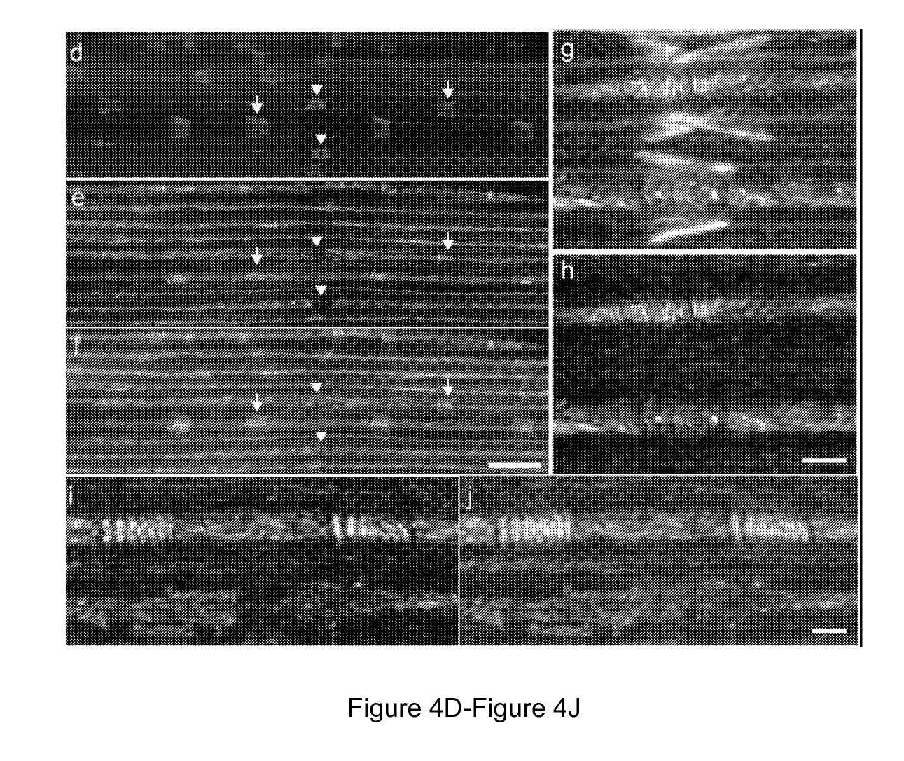

Using transgenic mice that express yellow fluorescent protein in a subset of layer V cortical pyramidal neurons (Thy1-YFPh), reflective processes that fully co-localized with the YFP fluorescent signal were occasionally observed, suggesting that the reflectance originated specifically from axons (FIG. 1H-FIG. 1J). However, only a subpopulation of reflective processes colocalized with YFP-labeled axons (FIG. 1H-FIG. 1J), suggesting that they represented only those with myelination, given the known reflective properties of myelin (Fille and Peuker, 2000, J. Pathol. 190:635-8; Xiao et al., 2006, Brain Res. 1077:81-9). To confirm this, it was sought to label cortical myelin fluorescently using the myelin specific dye Fluoromyelin (FM), although this dye had not been previously used intra-vitally. FM was applied topically to the cortical surface through a cranial window and it was found that it robustly labeled the compact myelin sheath of superficial axons as evidenced by the typical tubular appearance (FIG. 2B). Comparing the FM fluorescence to the reflection signal, it was found that 100% of FM-labeled fibers were also reflective (FIG. 2A-FIG. 2C). Additionally, YFP-labeled dendrites and unmyelinated axons did not demonstrate any reflectance (FIG. 2D-FIG. 2G) as was the case for astrocytes and microglia (FIG. 7). Furthermore, it was found that axonal segments that did not have FM labeling, for example at some axonal bifurcations (FIG. 2H-FIG. 2K), which have been described to lack myelin (Ha, 1970, J. Comp. Neurol. 140:227-40; Waxman et al., 1975, Neurosci. Lett. 1,251-6), were not reflective, even though the axon was normal throughout as evidenced by the intact YFP signal. In addition, when imaging in vivo the mouse sciatic nerve using SCoRe, it was found that individual FM labeled axons were clearly visible (FIG. 2L) but lacked reflection at nodes of Ranvier (FIG. 2L-FIG. 2N, FIG. 4D-FIG. 4F, and FIG. 4I-FIG. 4J). This data demonstrates that myelin is the source of fiber reflection in both the cortex and peripheral nerve.

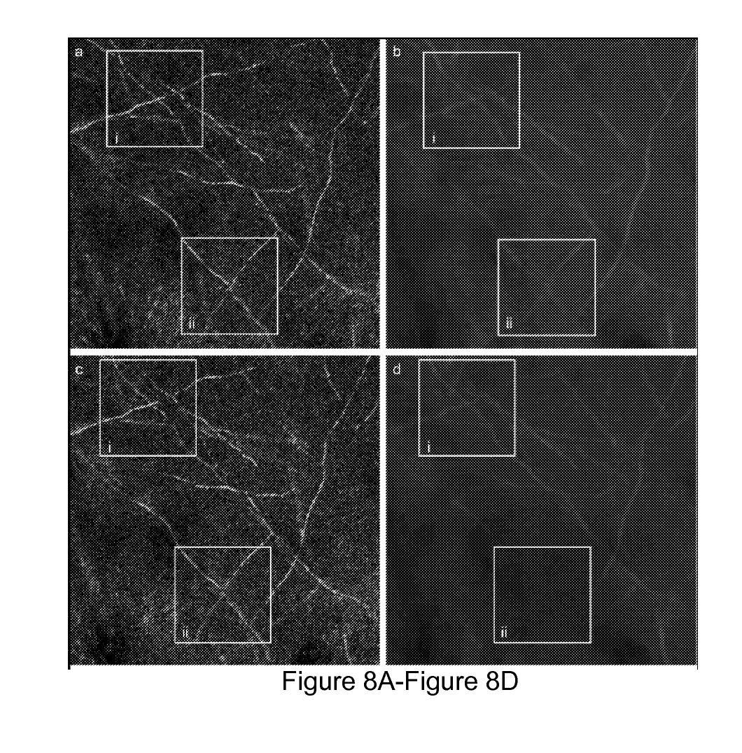

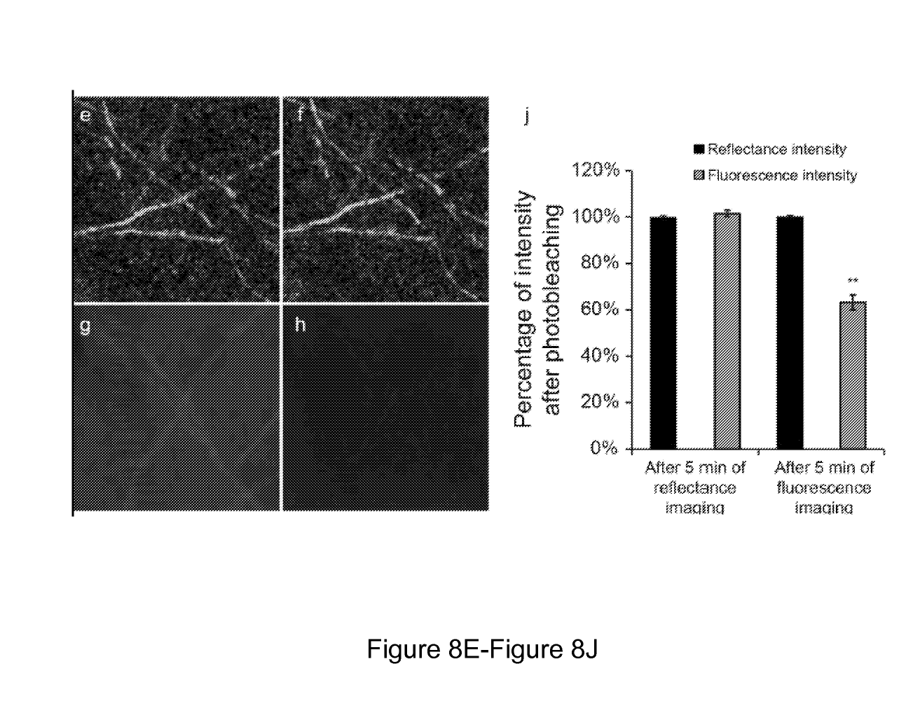

Although with SCoRe, the wavelengths used are shorter and light collection less efficient than with two-photon microscopy, a commonly used deep tissue imaging technique, it was surprising to see that SCoRe was able to detect myelinated axons as deep as 400 μm below the pial surface and using low laser intensities (˜300 μW at the sample). While not wishing to be bound by any particular theory, the likely explanation for this property is that myelin is highly reflective, making light scattering and collection efficiency less critical for SCoRe than for fluorescence imaging. Thus SCoRe can provide high resolution images of deep myelinated axons with low laser intensities, allowing repeated imaging at high zooms with virtually no photo-bleaching of adjacent fluorescent structures (FIG. 8).

### In Vivo Time-Lapse SCoRe Reveals Distinct Patterns of Cortical Myelination in Postnatal and Adult Mice

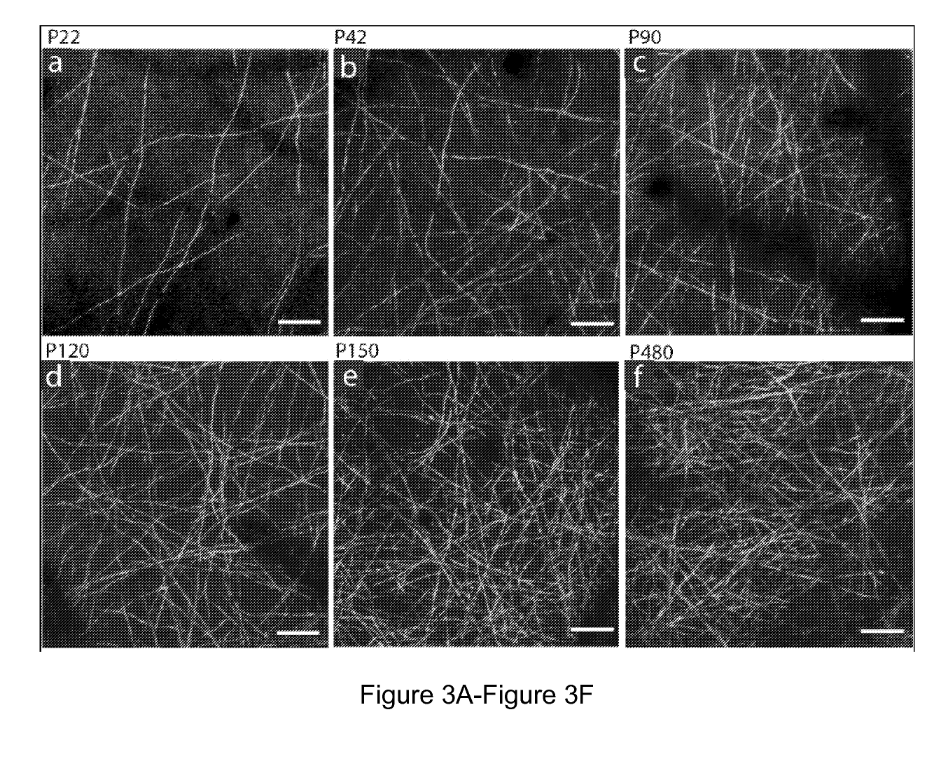

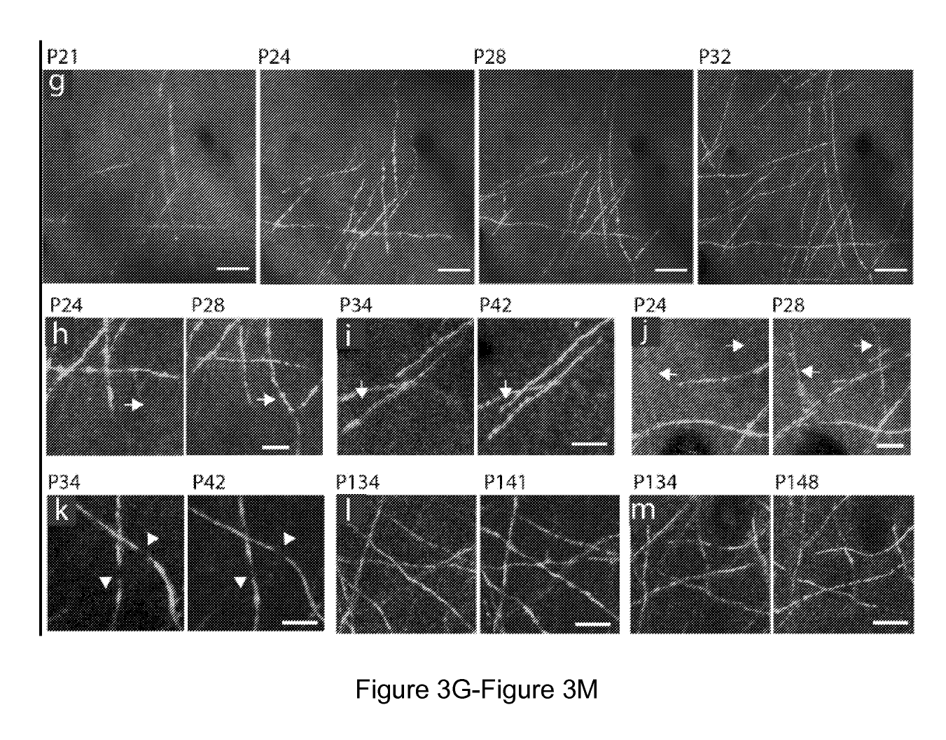

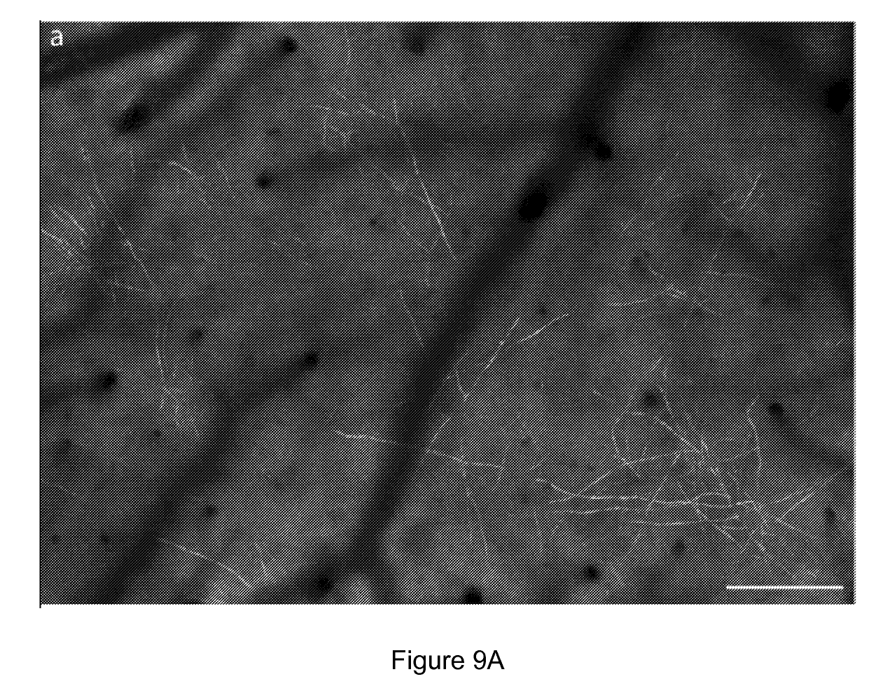

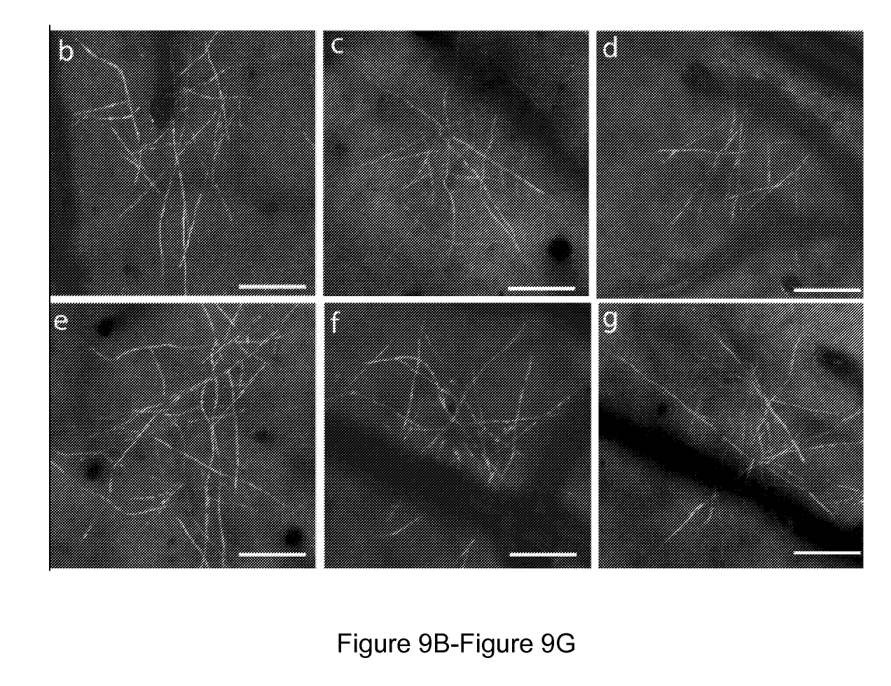

To test the ability to perform time-lapse imaging of cortical myelinated axons, SCoRe was used to image mice at various ages through a thinned skull preparation (Yang et al., 2010, Nat. Protoc. 5:201-8). Myelination in the mouse cortex begins in the second postnatal week and continues into adulthood (Jacobson, 1963, J. Comp. Neurol. 121:5-29). Consistent with this, it was found that young mice lacking cortical myelin had neither fibers that stained with FM nor reflective fibers. Around postnatal days 18 to 21 however, reflective fibers were first detected in the superficial cortex (FIG. 9). Interestingly, at this age these fibers appeared in individual patches which closely resembled the morphology of myelinating oligodendrocytes, with patch diameters ranging from 200 to 300 μm, similar to what has been shown with fluorescence reporters of mouse cortical oligodendrocytes (Chong et al., 2012, Proc. Natl. Acad. Sci. U.S.A. 109:1299-304) (FIG. 9). At subsequent developmental stages and into adulthood, a steady increase in the density of reflective axons in layers I-II was observed (FIG. 3A-FIG. 3F). Little is known about the dynamic changes in myelin over time on the cellular scale in vivo; therefore SCoRe was used to re-image the same regions in a mouse over several weeks. During a relatively short period of time, cortical sub-regions that became myelinated, likely due to the maturation of individual oligodendrocytes, were able to be visualized (FIG. 3G). Further, individual axons which became newly myelinated, (FIG. 3H-FIG. 3J) stable unmyelinated regions that resembled nodes of Ranvier, (FIG. 3K) and unchanged myelinated regions in older animals (FIG. 3L-FIG. 3M), were all able to be observed. These examples show how myelin formation, stability and specific subregions of the axon can be dynamically investigated in mouse cortex during development and in the adult using SCoRe. The technique could be especially informative and powerful when combined with fluorescently labeled structures of interest, such as oligodendrocyte progenitor cells and mature myelinating oligodendrocytes.

### SCoRe Generates a Multicolor Reflectance Spectrum that Highlights Unique Myelinated Axon Features

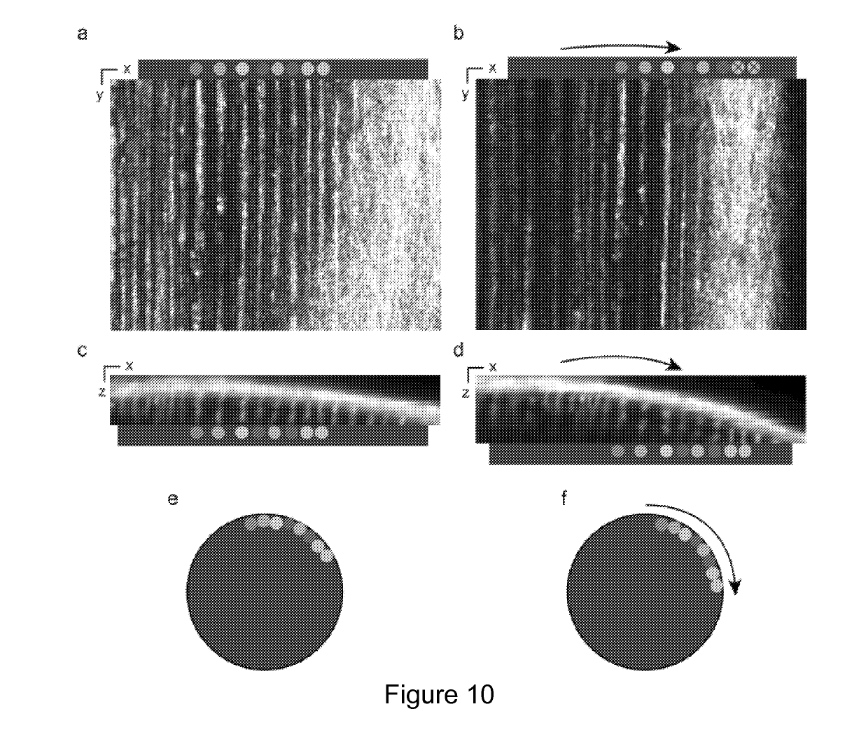

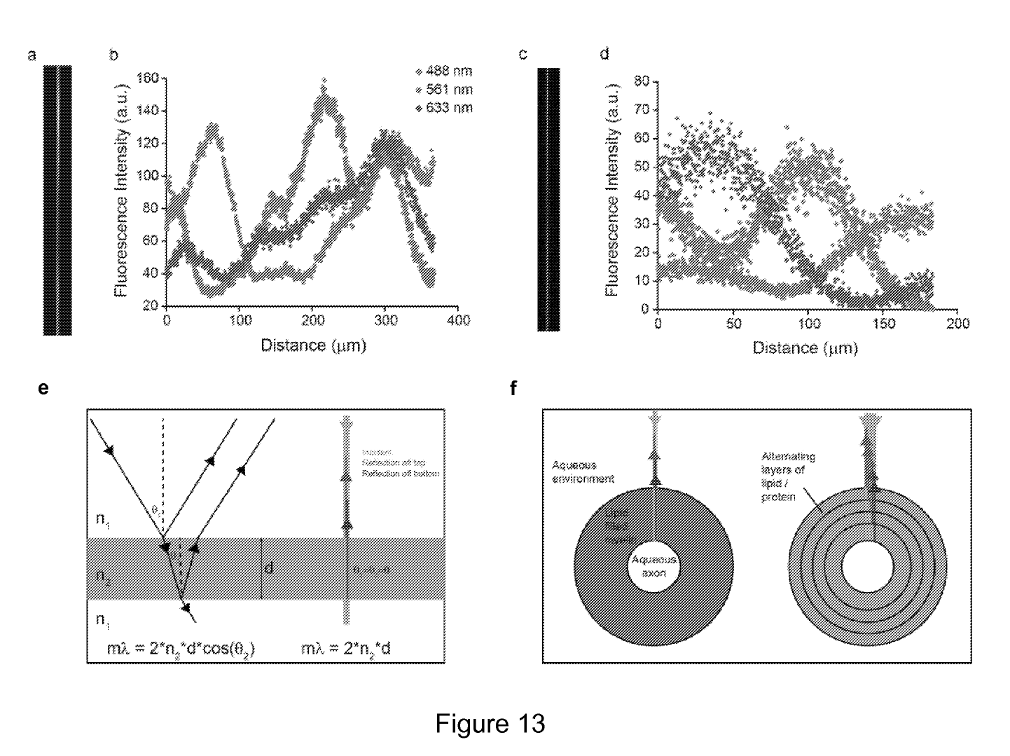

The simultaneous use of the reflectance signal from various lasers not only produced a contiguous myelinated axonal image (FIG. 1G, cyan), but when imaging at high zoom appeared as a multicolor speckle pattern (FIG. 1F). Remarkably, despite this heterogeneous speckle, it was noticed in the spinal cord and sciatic nerve, that individual axons also had a unique predominant reflectance color that allowed us to distinguish them from adjacent axons despite the high axonal density (FIG. 4). This unique color pattern was intrinsic to the myelinated fiber and was not significantly affected by axial rotations of the sample (FIG. 10). This signal is likely to originate predominately from abrupt changes in refractive index between the mostly aqueous neural tissue (Binding et al., 2011, Opt. Express 19:4833-47) and the lipid-rich myelin which causes highly efficient light reflection. The multicolor nature of this reflection is likely due to a phenomenon analogous to thin-film interference (Macleod, H. A. Thin-Film Optical Filters. Sciences New York 668 (Institute of Physics Publishing: 2001).doi:10.1887/0750306882) (FIG. 13), which leads to constructive and destructive interference of particular wavelengths, depending on differences in the thickness of the reflective surface (See FIG. 13 for experimental evidence of the effect of the thickness of an axon-like glass fiber on its reflectance color). Therefore, in the case of myelin, the number, thickness, and relative composition of the membranous layers may have a direct impact on the reflected spectrum. While not wishing to be bound by any particular theory, the most likely explanation for the big differences in the overall color of adjacent axons in the peripheral nerve and spinal cord is the large inter-axonal variability in diameter and degree of myelination in contrast with those in the superficial cortex. Regardless of the precise mechanism, the color reflection observed allows the unambiguous identification and tracing of individual axons over relatively long distances in peripheral nerves and spinal cord, and the differences in colors likely contain information about axonal and myelin diameter or composition (FIG. 4).

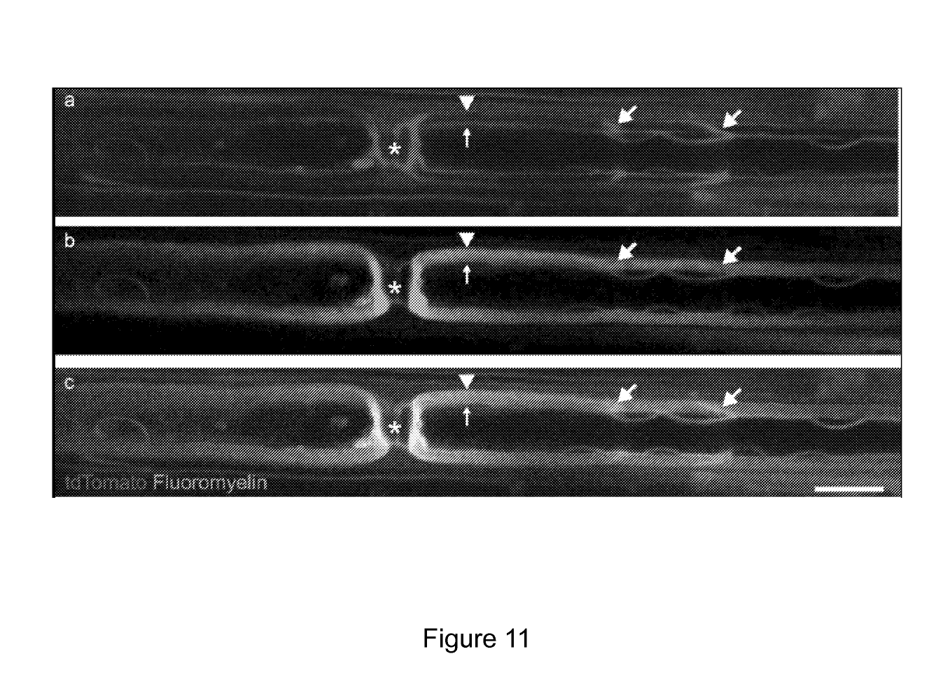

More detailed visualization of the colors on individual axons in the sciatic nerve revealed a peculiar periodic vertical multicolor reflection pattern that occurred at irregular intervals of approximately 20-60 μm (FIG. 4D-FIG. 4J). To further investigate the cellular origin of this reflection it was examined whether mice expressing ubiquitous fluorescence would be useful for simultaneously imaging myelin and its reflection in peripheral nerves. Indeed, when mT/mG transgenic mice, which express tdTomato in cell membranes were imaged; highly detailed images of myelin layers were surprisingly observed (FIG. 11). Also observed was the typical oblique appearance of Schmidt-Lanterman incisures (SLI), which are cytoplasmic channels, within the otherwise highly compact myelin (FIG. 4D, arrows). Remarkably, the periodic vertical reflection areas that were observed were completely colocalized with fluorescent SLIs. These incisures are important structures within myelin that are thought to be critical for cytoplasmic and molecular flow along myelin layers (Meier et al., 2004, J Neurosci, 24: 3186-3198). SCoRe is the only label-less technique that can unambiguously image these structures, opening the possibility of studying them in vivo in the context of a variety of pathologies (Reynolds and Heath, 1995, J Anat, 187: 369-378). Furthermore, the combination of mice with fluorescently labeled myelin and SCoRe imaging is likely to be a powerful tool for studying these structures experimentally in vivo.

### SCoRe is Capable of Detecting Acute and Chronic Myelin Pathology

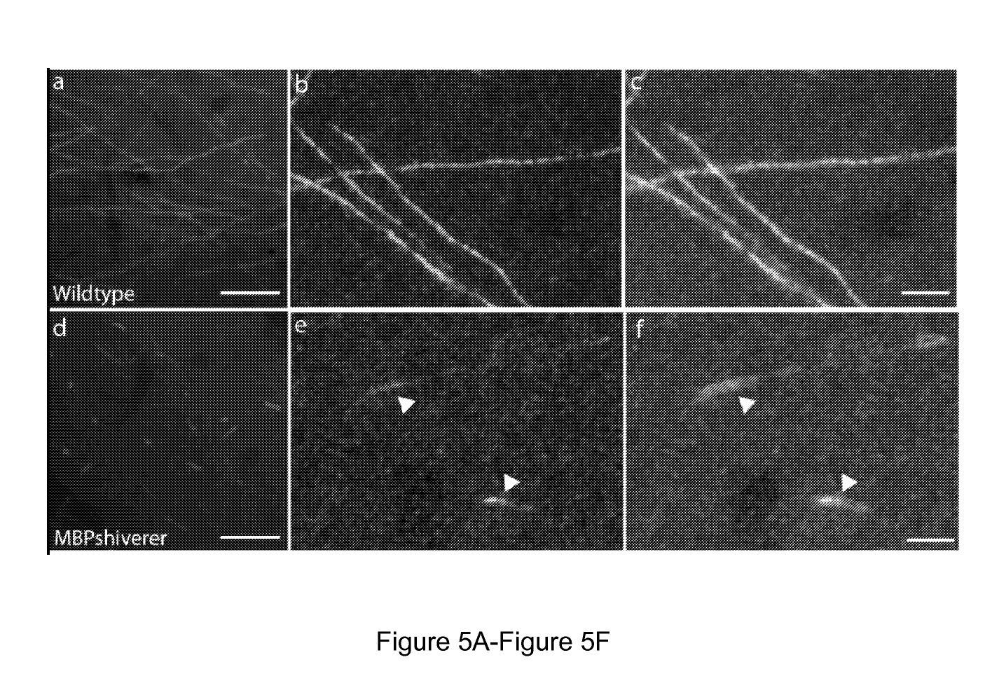

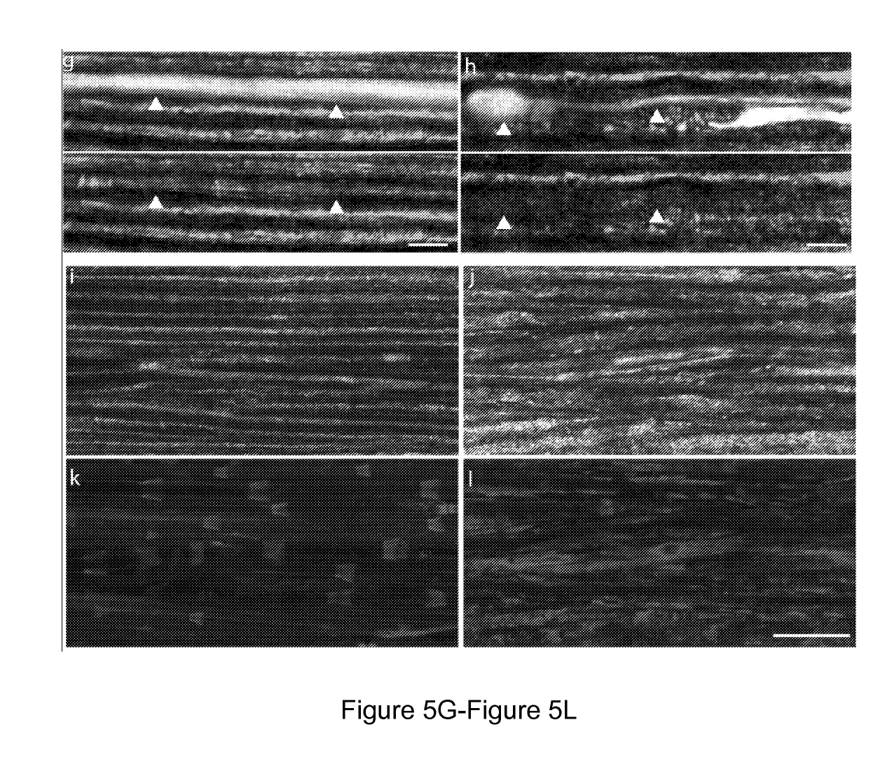

First, it was sought to determine if SCoRe was able to detect differences in patterns of myelination in shiverer mice, a well-known model in which mutation of the myelin basic protein (MBP) gene prevents the effective formation of compact myelin. In vivo SCoRe imaging of the cortex of shiverer mice showed a marked paucity of reflective fibers throughout (FIG. 5E). Occasionally, small scattered segments of reflectance were found, but no continuous axons were observed (FIG. 5E). To further test if these were actually areas lacking myelin, the cortical surface was labeled by topical application of fluoromyelin (FM). Indeed, shiverer mice had a severely reduced FM labeling but those scattered areas of reflection that were observed uniformly colocalized with patches of FM labeling (FIG. 5D-FIG. 5F). This not only demonstrates that the bright reflectance signal is entirely dependent on the presence of compact myelin but also shows that SCoRe is a very sensitive method for detecting myelination defects.

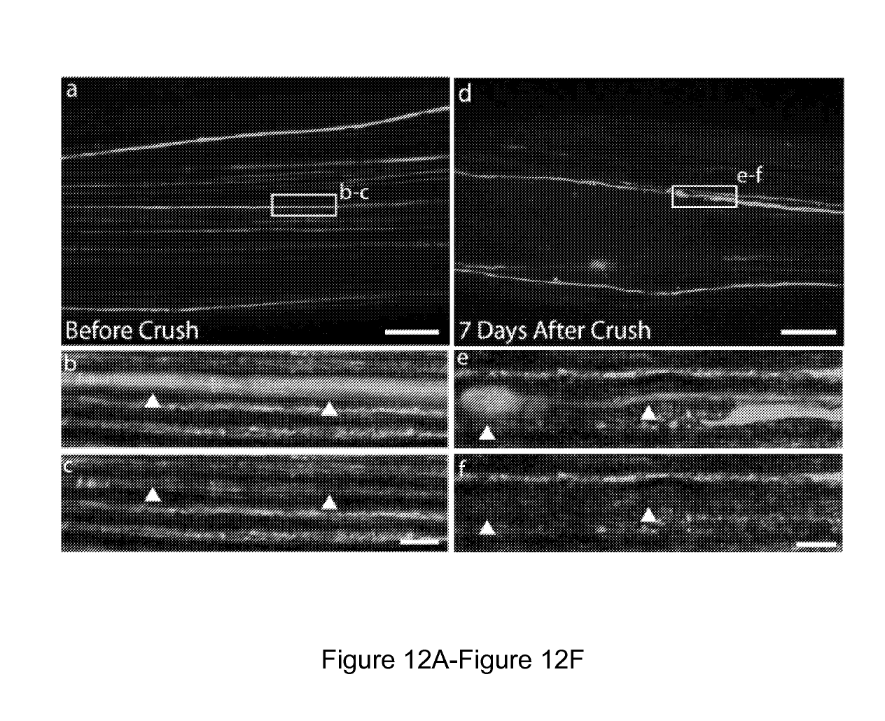

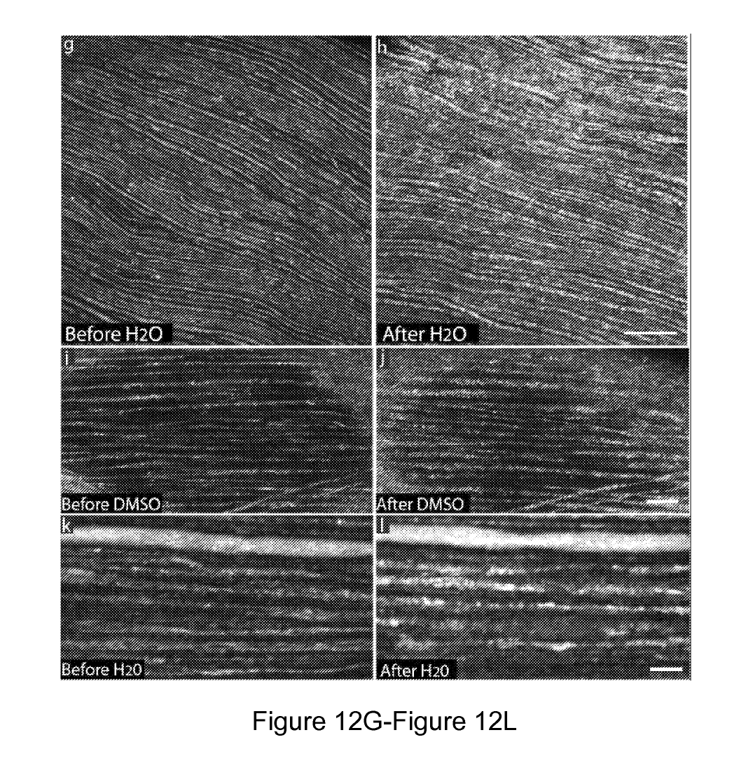

Given that the reflection color spectrum is likely to be affected by subtle changes in myelin composition and integrity, it was examined whether SCoRe would be a sensitive method for early detection of myelin disruption. Indeed, when the sciatic nerve was exposed to the demyelinating agent lysophosphatidylcholine (LPC) while imaging the nerve in vivo, changes manifested as a loss of a clear color pattern in the affected area, was observed within hours. Similar color changes were observed after exposure to DMSO, which changes the membrane phospholipid composition and with a hypotonic solution which causes myelin swelling (FIG. 5I-FIG. 5L and FIG. 12). These data demonstrate that SCoRe can be used to detect very early myelin pathology in vivo.

As myelin damage becomes more severe, the reflected colors are less relevant as reflection may completely disappear. For example, a sciatic nerve crush injury was performed in a Thy1-YFPh mouse, which damages axons and disrupts myelin in a specific area of the nerve. In vivo imaging of the damaged nerve with both SCoRe and confocal fluorescence, revealed that regenerating unmyelinated YFP-labeled axons were not reflective 7 days post-injury (FIG. 5G-FIG. 5H and FIG. 12), although some adjacent axons were still myelinated. This demonstrates that SCoRe can be used to determine if and when damaged or regenerating axons are remyelinated.

### Imaging the Human Brain with SCoRe

Finally, SCoRe was performed on a fixed explant of human tissue to determine if myelinated fibers could be detected in an intact human cortex. The tissue was oriented in order to image the surface of the cortex into the superficial gray matter mimicking the situation for in vivo imaging of mouse brain (FIG. 6A). Consistent with the images acquired from live mouse tissue, a network of relatively sparse multicolored fibers that were highly reflective was observed (FIG. 6B-FIG. 6E). These fibers were easily detected with laser intensities comparable to those used in mouse brain. Next, fluoromyelin was applied to the pial layer of the human cortex in a similar fashion used for in vivo labeling in mouse and it was found that fluoromyelin specifically labeled the reflective fibers (FIG. 6F-FIG. 6J). This was consistent with images acquired from mouse tissue and confirmed that the reflective fibers in human tissue are indeed myelinated axons. These data show the feasibility of imaging myelinated fibers in intact human tissue with high resolution, no dyes, and very little photo-bleaching (FIG. 8) and potential photo-toxicity.

### Imaging of Myelin

In vivo optical fluorescence microscopy which allows visualization of the same structures repeatedly over time, has demonstrated to be invaluable in understanding the structural and functional plasticity of a variety of cells in the nervous system during development, aging and pathology (Grutzendler et al., 2002, Nature 420:812-6; Davalos et al., 2005, Nat. Neurosci. 8:752-8; Harb et al., 2012, J. Cereb. Blood Flow Metab. 33:146-156). Described herein is the development of a new technique that allows label-less high-resolution in vivo optical imaging of myelinated axons using spectral confocal reflectance microscopy (SCoRe). Because SCoRe requires only a modern confocal microscope with spectral detection capabilities, which is widely available and routinely used throughout the world, this technique could have wide applications in preclinical animal studies of a large number of pathological conditions affecting myelin. In addition, given its versatility and sensitivity to changes in myelin properties, SCoRe could be used as a novel diagnostic tool for human peripheral and central myelin disorders.

With SCoRe, images are generated by laser reflection resulting from large changes in refractive index from the mostly aqueous brain parenchyma to the lipid-rich myelin. Merging the detected reflections of multiple laser wavelengths, which individually produce a segmented image pattern, generates a complementary and continuous high-resolution axonal image (FIG. 1 and FIG. 6). Several lines of evidence demonstrate that the bright reflected signals originate exclusively from myelinated axons: first, when the cortex of mice that express yellow fluorescent protein in neurons was imaged, none of the dendrites and only a subpopulation of axons were reflective (FIG. 1 and FIG. 2). Second, when the myelin specific dye, fluoromyelin (FIG. 2) was applied to the cortex, it was observed that only axons that became labeled generated a reflection signal. Third, even within a reflective axon there were small segments that did not reflect, which precisely matched areas of no fluoromyelin staining (FIG. 2), consistent with nodes of Ranvier (Poliak and Peles, 2003, Nat Rev Neurosci, 4: 968-980) or other unmyelinated axonal sections. Interestingly, in the cortex, it was observed that many unmyelinated portions of axons tended to be located at sites of axonal bifurcations (FIG. 2). Such unmyelinated areas have previously been observed by electron microscopy (Ha, 1970, J. Comp. Neurol. 140:227-40; Waxman et al., 1975, Neurosci. Lett. 1:251-6), however, their identification in the cortex in vivo has never been reported and their function remains unknown. Fourth, the density of brain reflective fibers increased rapidly after the 3rd postnatal week and stabilized after the 3-4^{th} postnatal month, consistent with previously published histological data on the time course of mouse cortical myelination (Jacobson, 1963, J. Comp. Neurol. 121:5-29) (FIG. 3). Fifth, the reflection signal was markedly reduced in shiverer mice, which have a severe defect in the formation of compact myelin (FIG. 5).

The precise mechanism for the multicolor reflection pattern is not clear but, without wishing to be bound by any particular theory, it likely relates to the principle of thin-film interference (Macleod, H. A. Thin-Film Optical Filters. Sciences New York 668 (Institute of Physics Publishing: 2001).doi:10.1887/0750306882). Similar to dichroic mirrors which have alternating layers of optical coatings with different refractive indices, myelin is composed of many layers of lipid-rich sheaths. This layered structure is likely to lead to constructive and destructive light interference selectively reinforcing certain reflected wavelengths and suppressing others (FIG. 13). Like with dichroic mirrors, the thickness and number of layers may determine the wavelengths that are preferentially reflected but in the case of myelin, further variability may occur due to irregularities in myelin sheath thickness, lipid composition and several other local cellular variables. Interestingly, it was found that in addition to the observed focal color patchiness, individual axons displayed a unique overall color signature. This was especially prominent in axons of the sciatic nerve and spinal cord but less noticeable in the cortex. While not wishing to be bound by any particular theory, the most likely explanation for this phenomenon stems from the fact that axons in peripheral nerves and spinal cord are larger and have a wide inter-axonal variability in diameters and degree of myelination in contrast to the smaller and thinly myelinated axons in the superficial cortex. Importantly, these unique color features of individual axons in the spinal cord and peripheral nerve facilitated their identification and tracing during time-lapse imaging despite the high density of adjacent processes (FIG. 4), similar to methods such as brainbow (Livet et al., 2007, Nature 450:56-62) or Diolistic neuronal labeling (Gan et al., 2000, Neuron 27:219-25) but without the need for dyes or genetically encoded fluorescent proteins. In addition, a striking periodic multicolor pattern of reflection derived specifically from Schmidt-Lanterman incisures was also found in peripheral nerve axons. These cellular structures, which have not been extensively studied, have recently gained interest as they are thought to constitute important cytoplasmic channels that facilitate the transport of metabolites and degradation products throughout the myelin sheath (Meier et al., 2004, J. Neurosci. 24:3186-98; Reynolds and Heath, 1995, J. Anat. 187:369-78). While the in vivo visualization of incisures was introduced using fluorescence imaging in mT/mG mice, SCoRe is the only technique that can identify these structures in vivo without the need for fluorescent labeling. Such capability may open new avenues for understanding the physiology of these myelin structures and their role in neuropathology.

Another important feature of the multicolor reflection is its high sensitivity to acute alterations in myelin structure and composition. For example, peripheral nerve exposure to Dimethylsulfoxide (DMSO) which is known to alter the composition of myelin phospholipids (Kirschner and Caspar, 1975, Proc. Natl. Acad. Sci. U.S.A. 72:3513-7) or exposure to a hypo-osmolar solution, which likely causes myelin swelling, increased the overall reflectivity of all wavelengths and eliminated many of the color differences between axons. Furthermore, application of the demyelinating agent lysophosphatidylcholine (LPC) also disrupted the reflection color pattern. Finally, SCoRe was also sensitive to chronic developmental myelin abnormalities as evidenced by the clear detection of myelin deficits in shiverer mice. Thus SCoRe is a powerful method to identify early changes and chronic defects in myelin composition or structure.

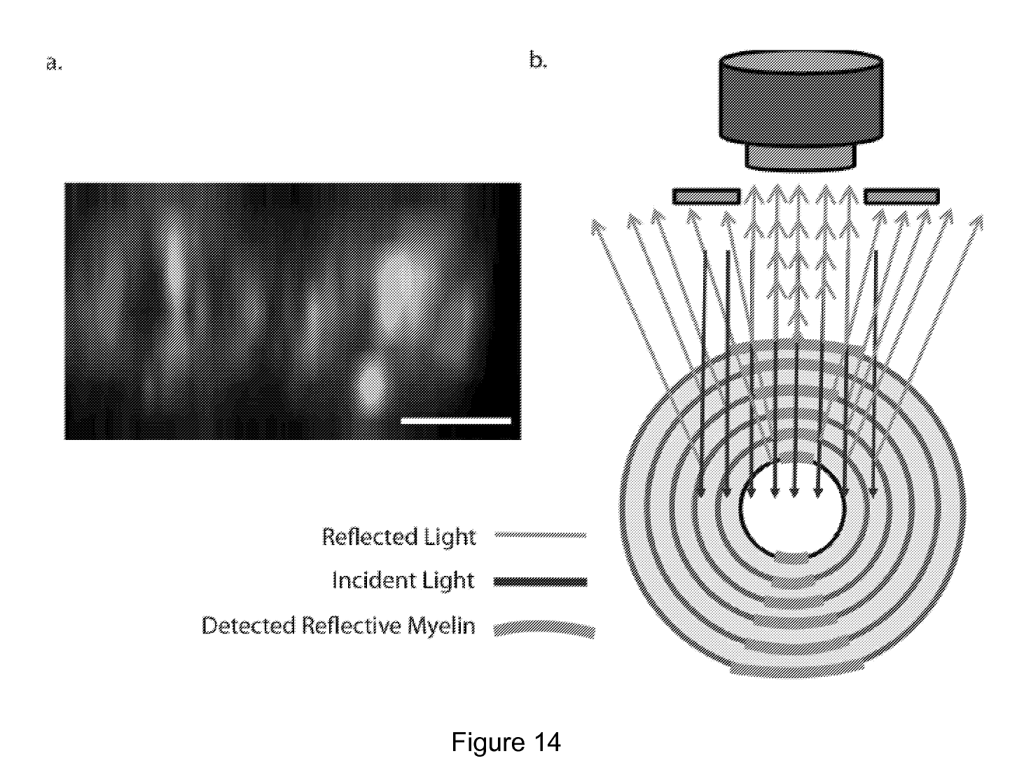

Several methods are currently available for in vivo imaging of myelin and myelin producing cells. Confocal and two photon fluorescence microscopy has been reported using genetically encoded fluorescent markers in zebrafish oligodendrocytes (Kirby et al., 2006, Nat. Neurosci. 9:1506-11) and mouse peripheral nerve Schwann cells. Using these fluorescent cellular reporters, however, it is not easy to trace individual myelinated axons or determine when oligodendrocyte cellular processes near an axon have established a mature compact myelin sheath. SCoRe, however could complement fluorescence microscopy by providing a clear traceable image of the axon during the process of compact myelin sheath formation by adjacent oligodendrocyte processes. Several techniques have been developed that are capable of imaging myelin without the use of fluorescent labeling. These include optical Coherence Microscopy (OCM) (Ben Arous, 2011, J Biomed Opt, 16: 116012), Coherent Anti-Stokes Raman Scattering (CARS) (Fu et al, 2008, Opt. Express 16:19396-409; Imitola et al, 2011, J. Biomed. Opt. 16:021109; Gupta et al., 2012, Sci. Transl. Med. 4:155ra137) and third harmonic generation (THG) (Witte et al., 2011, Proc. Natl. Acad. Sci. U.S.A. 108:5970-5; Farrar et al., 2011, Biophys. J. 100:1362-71) which rely on the reflective, vibratory or anisotropic properties of lipid-rich myelin respectively. In general, these techniques have great potential but require complex setups or rarely available equipment. Because SCoRe uses a conventional confocal microscope with spectral detection, it is easy to implement and is very sensitive, allowing the generation of high signal to noise images of myelinated axons. Furthermore, SCoRe could potentially be used in combination with CARS and THG to generate a more comprehensive picture of the myelination status. Specifically, the spectral multicolor features of SCoRe, which provide unique information about the structural integrity of the underlying myelin, nodes of Ranvier and Schmidt-Lanterman incisures, could be complemented with the ability of THG and CARS to generate images of the actual myelin sheath that better match those of fluorescent dyes such as fluoromyelin (FIG. 14). While not wishing to be bound by any particular theory, FIG. 14 depicts a possible mechanism for the hour glass shape observed in the reflectance image when an myelinated axon is imaged orthogonally. The reflection (cyan arrows) from the incident light (black arrows) is detected through the confocal pinhole and PMT only from the bold portion of each layer of myelin. As the surface area of the myelin decreases with smaller diameter the area that is detected decreases at a given angle of reflection therefore as the diameter of the myelin sheath decreases the reflected signal detected similarly decreases. This phenomenon also occurs on the bottom layers of myelin (not shown for clarity) resulting in a reflected signal that originates from each myelin layer but appears to come from the axon if examined in the X-Y plane. That being said, it cannot be ruled out with 100% certainty that the reflected signal is not coming from the axon that is myelinated. It is possible, however unlikely, that something about an axon being myelinated changes the axon's reflective properties via intracellular changes in microtubules, organelles, protein expression, etc. This is thought to be highly unlikely for several reasons; first, lipid (the major component of myelin) is known to be highly reflective and much more so than other cellular components present in the tissue. Second, if the reflected signal was originating from the axon it would be expected that the widest/strongest signal to appear at the center of the axon which is the opposite of what we have observed. Third, Schmidt-Lanterman incisures (which are exclusively a structure within myelin) alter the reflected signal. Fourth, such changes in reflective properties of the axon are not likely to change so dramatically in small non-myelinated regions such as nodes of Ranvier and axonal bifurcations and to occur in sub-regions along an axon when it is being myelinated developmentally.

Although two-photon microscopy (TPM) has been widely used for deep tissue fluorescence imaging with relatively minimal damage, it can still produce significant photo-bleaching and thermal injury (Davalos et al., 2005, Nat. Neurosci. 8:752-8) especially during repeated time-lapse imaging. On the other hand, because SCoRe is highly sensitive to myelin, it requires light levels that are substantially lower (on the order of 200-400 μW at the sample) than those used for conventional confocal fluorescence or two photon microscopy. Thus, SCoRe can be used for repeated imaging at high zooms with virtually no photo-toxicity or thermal injury (FIG. 3 and FIG. 8), making it ideal for in vivo use. Furthermore, longer wavelength Ti:sapphire lasers, can also be used to generate a reflection signal with SCoRe. Using such lasers would in theory allow SCoRe to achieve even greater tissue penetration than two photon microscopy as both incident and reflected lights would have infrared wavelengths which are relatively less scattering.

Using SCoRe, several novel observations were made in vivo: for the first time newly formed myelinated structures were tracked longitudinally in the living mammalian brain and it was found that myelination progresses rapidly over days (FIG. 3) but change is confined to isolated micro-regions (FIG. 3 and FIG. 9) likely to represent territories covered by individual oligodendrocytes. Second, areas were found lacking myelination at axonal bifurcations in the cortex. This under-investigated phenomenon, which was first described decades ago (Waxman et al., 1975, Neurosci. Lett. 1:251-6; Ha, 1970, J. Comp. Neurol. 140:227-40) is of unknown function but can now be studied in vivo with SCoRe. Third, for the first time imaging of Schmidt-Lanterman incisures in vivo (FIG. 4) was able to be done in the peripheral nerve and paranodes and nodes of Ranvier were able to be tracked over time in both the peripheral and central nervous systems (FIG. 3 and FIG. 4). Finally, it was demonstrated the feasibility of imaging the spinal cord with SCoRe, opening the possibility for studying spinal cord injury and regeneration.

Future modifications of SCoRe with longer wavelength infrared laser excitation, sensitive infrared detection capabilities (Hong et al., 2012, Nature Medicine 18:1841-1846) or potentially fiber-optic endoscopy, would significantly increase the imaging depth, and allow SCoRe imaging of human brain, spinal cord and peripheral nerve for medical reasons. For example, recently it has become possible to reconstitute myelin with engrafted neural stem cells in humans with severe leukodystrophies (Gupta et al., 2012, Sci. Transl. Med. 4:155ra137; Windrem et al., 2008, Cell Stem Cell 2:553-65). Additionally, it has been documented that subpial cortical demyelination is one of the earliest pathological events in multiple sclerosis (Bø, et al., 2003, J. Neuropathol. Exp. Neurol. 62:723-32; Lucchinetti et al., 2011, N. Engl. J. Med. 365:2188-97). The presently described technique, which allows for high resolution imaging in the intact cortex and spinal cord, could potentially be used for tracking the effective formation of myelin after engraftment or its degeneration in demyelinating disorders. Furthermore, conditions such as demyelinating, toxic and autoimmune peripheral neuropathies may trigger unique changes in nodes of Ranvier, Schmidt-Lanterman incisures and other myelin features which may be detectable by SCoRe. Therefore, this technique could become an important application for imaging peripheral nerves instead of tissue biopsy for diagnosing a variety of neuropathies. Thus, SCoRe is a versatile and powerful technique that adds significant capabilities to the toolbox for in vivo imaging of the central and peripheral nervous systems in a variety of animal models and potentially in humans.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method for imaging myelin comprising
administering a multi-wavelength laser light to a region of interest in a subject;
collecting the reflected light from the sample, wherein the collected reflected light is indicative of myelin in the region of interest; and
detecting the reflected light at each wavelength of the administered multi-wavelength laser light.

2. The method of claim 1, further comprising constructing a plurality of single-wavelength images, each single-wavelength image based on the detected reflected light at each wavelength, and combining the plurality of single-wavelength images to form a multi-wavelength image.

3. The method of claim 1, wherein the method identifies Nodes of Ranvier in a myelinated axon.

4. The method of claim 1, wherein the method identifies Schmidt-Lanterman incisures in a myelinated axon.

5. The method of claim 1, wherein the region of interest is in a body tissue selected from the group consisting of brain, spinal cord, and peripheral nerve.

6. The method of claim 1, wherein the method detects myelin at a depth of about 400 μm.

7. The method of claim 1, wherein the intensity of the multi-wavelength laser light is about 200-400 μW.

8. The method of claim 1, wherein the multi-wavelength laser light comprises at least three different wavelengths.

9. The method of claim 1, wherein the multi-wavelength laser light comprises the wavelengths of 488 nm, 561 nm, and 633 nm.

10. The method of claim 1, wherein the method identifies the subject as having a myelin-related disorder selected from the group consisting of multiple sclerosis, transverse myelitis, leukodystrophies, Guillain-Barre syndrome, diabetic neuropathy, inflammatory neuropathies, inherited demyelinating conditions, Charcot-Marie Tooth, cancer related neuropathies, and toxic neuropathies.

11. The method of claim 1, wherein the method identifies the location of myelinated axons during surgery.

12. The method of claim 1, wherein the method identifies a tumor as being formed of or caused by myelin producing cells.

13. The method of claim 1, wherein the method determines the aggressiveness of a tumor.

14. A system for imaging myelin comprising
at least one light source, wherein the at least one light source emits a plurality of beams of light, each beam having a different wavelength; thereby generating a plurality of beams of a plurality of wavelengths;
a lens for guiding the plurality of beams to a sample; and
a plurality of photodetectors, wherein each photodetector is configured to detect the reflected light of one of the wavelengths of the plurality of beams.

15. The system of claim 14, wherein the at least one light source comprises a plurality of light sources, each configured to emit a beam of light.

16. The system of claim 14, wherein the at least one light source comprises at least one light source selected from the group consisting of an Argon laser, a Diode Pumped Solid State (DPSS) laser, a Helium/Neon laser, a supercontinuum (white-light) laser, a laser diode, and a pulsed Ti-Sapphire laser.

17. The system of claim 14, wherein the system comprises an Acousto-Optical Tunable Filter.

18. The system of claim 14, wherein the system comprises a 30/70 partially reflective mirror.

19. The system of claim 14, wherein the system comprises a scanner.

20. The system of claim 14, wherein the plurality of photodetectors comprise a photomultiplier tube detector.
